SEQUENCE LISTING (37 sequences)

SEQ ID NO: 1 - DNA, 471 bp, Artificial Sequence
  Chemically Synthesized, Human IL-36 alpha full length
gaaaaagccctgaagattgacacccctcagcagggctctatccaggacatcaaccaccgggtgtgggtgctgcaggatcagaccctgatcgccgtgccacggaaggacagaatgagccccgtgaccatcgccctgatctcctgcaggcacgtggagacactggagaaggatcgcggcaatcccatctacctgggcctgaacggcctgaatctgtgcctgatgtgcgcaaaagtgggcgaccagcctaccctgcagctgaaggagaaggacatcatggatctgtacaaccagcccgagcctgtgaagtccttcctgttttatcactctcagagcggcaggaattccacattcgagtctgtggccttccctggctggtttatcgccgtgagctccgagggcggctgtccactgattctgactcaggaactggggaaggctaatactaccgattttggactgactatgctgttt

SEQ ID NO: 2 - protein, 157 aa, Artificial Sequence
  Chemically Synthesized, Human IL-36 alpha full length
EKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 3 - DNA, 459 bp, Artificial Sequence
  Chemically Synthesized, Human IL-36 alpha truncated
aagattgatacacctcagcagggaagcattcaggacatcaaccaccgggtgtgggtgctgcaggatcagaccctgatcgccgtgccacggaaggacagaatgagccccgtgaccatcgccctgatctcctgcaggcacgtggagacactggagaaggatcgcggcaatcccatctacctgggcctgaacggcctgaatctgtgcctgatgtgcgcaaaagtgggcgaccagcctaccctgcagctgaaggagaaggacatcatggatctgtacaaccagcccgagcctgtgaagtctttcctgttttatcactctcagagcggcaggaattccacattcgagtctgtggccttccctggctggtttatcgccgtgagctccgagggcggctgtccactgattctgacccaggaactgggcaaggctaataccactgacttcggactgactatgctgttt

SEQ ID NO: 4 - protein, 153 aa, Artificial Sequence
  Chemically Synthesized, Human IL-36 alpha truncated
KIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 5 - DNA, 489 bp, Artificial Sequence
  Chemically Synthesized, Human IL-36 beta full length
aacccacagcgagaggcagcccccaaaagctacgccatccgggactctagacagatggtgtgggtgctgtccggcaactctctgatcgcagcccctctgagccggagcatcaagcctgtgaccctgcacctgatcgcctgtcgcgacacagagttctccgataaggagaagggcaatatggtgtacctgggcatcaagggcaaggatctgtgcctgttttgtgccgagatccagggcaagcccaccctgcagctgaagctgcagggcagccaggacaacatcggcaaggatacctgctggaagctggtgggcatccacacatgtatcaatctggacgtgcgggagtcctgcttcatgggcacactggatcagtggggcatcggcgtgggcaggaagaagtggaagagctcctttcagcaccaccacctgcgcaaaaaagacaaggacttcagctctatgagaactaacattgggatgcctgggaggatg

SEQ ID NO: 6 - protein, 163 aa, Artificial Sequence
  Chemically Synthesized, Human IL-36 beta full length
NPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 7 - DNA, 480 bp, Artificial Sequence
  Chemically Synthesized, Human IL-36 beta truncated
agggaagccgcccccaaaagctacgctatcagggactctcggcagatggtgtgggtgctgtccggcaactctctgatcgcagcccctctgagccggagcatcaagcctgtgaccctgcacctgatcgcctgtagagacacagagttctccgataaggagaagggcaatatggtgtacctgggcatcaagggcaaggatctgtgcctgttttgtgccgagatccagggcaagcccaccctgcagctgaagctgcagggcagccaggacaacatcggcaaggatacctgctggaagctggtgggcatccacacatgtatcaatctggacgtgagggagtcctgcttcatgggcacactggatcagtggggaatcggagtgggacgcaagaagtggaagagctcctttcagcaccaccacctgaggaagaaggataaagatttcagcagtatgcggactaacattgggatgccagggaggatg

SEQ ID NO: 8 - protein, 160 aa, Artificial Sequence
  Chemically Synthesized, Human IL-36 beta truncated
REAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 9 - DNA, 504 bp, Artificial Sequence
  Chemically Synthesized, Human IL-36 gamma full length
agaggaacccccggagatgctgatggaggaggacgggccgtgtaccagagcatgtgcaagcctatcaccggcacaatcaacgacctgaatcagcaagtgtggaccctgcagggacagaacctggtggcagtgccaaggtctgatagcgtgacccccgtgacagtggccgtgatcacctgtaagtatccagaggccctggagcagggacgcggcgaccctatctacctgggcatccagaatccagagatgtgcctgtattgtgagaaagtgggcgagcagccaaccctgcagctgaaggagcagaagatcatggatctgtacggccagcccgagcctgtgaagcccttcctgttttatcgggccaagacaggcagaacctccacactggagtctgtggccttccccgactggtttatcgccagctccaagagggatcagcctatcatcctgacctctgaactggggaaatcctacaatactgcttttgaactgaacattaacgac

SEQ ID NO: 10 - protein, 168 aa, Artificial Sequence
  Chemically Synthesized, Human IL-36 gamma full length
RGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 11 - DNA, 456 bp, Artificial Sequence
  Chemically Synthesized, Human IL-36 gamma truncated
agcatgtgcaagcctattactgggactatcaatgacctgaaccagcaagtgtggaccctgcagggccagaatctggtggccgtgccaaggtctgatagcgtgacccccgtgacagtggccgtgatcacatgcaagtaccctgaggccctggagcagggacgcggcgaccctatctacctgggcatccagaacccagagatgtgcctgtattgtgagaaagtgggcgagcagccaaccctgcagctgaaggagcagaagatcatggatctgtacggccagcccgagcctgtgaagcccttcctgttttatcgggccaagacaggcagaacctccacactggagtctgtggccttccccgactggtttatcgccagctccaagcgggatcagcctatcattctgacctccgaactggggaagtcctacaatacagcctttgaactgaacattaacgac

SEQ ID NO: 12 - protein, 152 aa, Artificial Sequence
  Chemically Synthesized, Human IL-36 gamma truncated
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 13 - DNA, 531 bp, Artificial Sequence
  Chemically Synthesized, Human IL-36 alpha full length
atggactggacttggattctgttcctggtcgccgccgcaactagagtgcattccgaaaaagccctgaagattgacacccctcagcagggctctatccaggacatcaaccaccgggtgtgggtgctgcaggatcagaccctgatcgccgtgccacggaaggacagaatgagccccgtgaccatcgccctgatctcctgcaggcacgtggagacactggagaaggatcgcggcaatcccatctacctgggcctgaacggcctgaatctgtgcctgatgtgcgcaaaagtgggcgaccagcctaccctgcagctgaaggagaaggacatcatggatctgtacaaccagcccgagcctgtgaagtccttcctgttttatcactctcagagcggcaggaattccacattcgagtctgtggccttccctggctggtttatcgccgtgagctccgagggcggctgtccactgattctgactcaggaactggggaaggctaatactaccgattttggactgactatgctgttttgataa

SEQ ID NO: 14 - protein, 175 aa, Artificial Sequence
  Chemically Synthesized, Human IL-36 alpha full length
MDWTWILFLVAAATRVHSEKALKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 15 - DNA, 519 bp, Artificial Sequence
  Chemically Synthesized, Human IL-36 alpha truncated
atggactggacatggattctgtttctggtcgccgccgcaactcgcgtgcatagcaagattgatacacctcagcagggaagcattcaggacatcaaccaccgggtgtgggtgctgcaggatcagaccctgatcgccgtgccacggaaggacagaatgagccccgtgaccatcgccctgatctcctgcaggcacgtggagacactggagaaggatcgcggcaatcccatctacctgggcctgaacggcctgaatctgtgcctgatgtgcgcaaaagtgggcgaccagcctaccctgcagctgaaggagaaggacatcatggatctgtacaaccagcccgagcctgtgaagtctttcctgttttatcactctcagagcggcaggaattccacattcgagtctgtggccttccctggctggtttatcgccgtgagctccgagggcggctgtccactgattctgacccaggaactgggcaaggctaataccactgacttcggactgactatgctgttttgataa

SEQ ID NO: 16 - protein, 171 aa, Artificial Sequence
  Chemically Synthesized, Human IL-36 alpha truncated
MDWTWILFLVAAATRVHSKIDTPQQGSIQDINHRVWVLQDQTLIAVPRKDRMSPVTIALISCRHVETLEKDRGNPIYLGLNGLNLCLMCAKVGDQPTLQLKEKDIMDLYNQPEPVKSFLFYHSQSGRNSTFESVAFPGWFIAVSSEGGCPLILTQELGKANTTDFGLTMLF

SEQ ID NO: 17 - DNA, 549 bp, Artificial Sequence
  Chemically Synthesized, Human IL-36 beta full length
atggactggacatggattctgttcctggtcgcagccgccacaagagtgcattcaaacccacagcgagaggcagcccccaaaagctacgccatccgggactctagacagatggtgtgggtgctgtccggcaactctctgatcgcagcccctctgagccggagcatcaagcctgtgaccctgcacctgatcgcctgtcgcgacacagagttctccgataaggagaagggcaatatggtgtacctgggcatcaagggcaaggatctgtgcctgttttgtgccgagatccagggcaagcccaccctgcagctgaagctgcagggcagccaggacaacatcggcaaggatacctgctggaagctggtgggcatccacacatgtatcaatctggacgtgcgggagtcctgcttcatgggcacactggatcagtggggcatcggcgtgggcaggaagaagtggaagagctcctttcagcaccaccacctgcgcaaaaaagacaaggacttcagctctatgagaactaacattgggatgcctgggaggatgtgataa

SEQ ID NO: 18 - protein, 181 aa, Artificial Sequence
  Chemically Synthesized, Human IL-36 beta full length
MDWTWILFLVAAATRVHSNPQREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 19 - DNA, 540 bp, Artificial Sequence
  Chemically Synthesized, Human IL-36 beta truncated
atggactggacatggattctgtttctggtcgcagcagccacaagagtgcattcaagggaagccgcccccaaaagctacgctatcagggactctcggcagatggtgtgggtgctgtccggcaactctctgatcgcagcccctctgagccggagcatcaagcctgtgaccctgcacctgatcgcctgtagagacacagagttctccgataaggagaagggcaatatggtgtacctgggcatcaagggcaaggatctgtgcctgttttgtgccgagatccagggcaagcccaccctgcagctgaagctgcagggcagccaggacaacatcggcaaggatacctgctggaagctggtgggcatccacacatgtatcaatctggacgtgagggagtcctgcttcatgggcacactggatcagtggggaatcggagtgggacgcaagaagtggaagagctcctttcagcaccaccacctgaggaagaaggataaagatttcagcagtatgcggactaacattgggatgccagggaggatgtgataa

SEQ ID NO: 20 - protein, 178 aa, Artificial Sequence
  Chemically Synthesized, Human IL-36 beta truncated
MDWTWILFLVAAATRVHSREAAPKSYAIRDSRQMVWVLSGNSLIAAPLSRSIKPVTLHLIACRDTEFSDKEKGNMVYLGIKGKDLCLFCAEIQGKPTLQLKLQGSQDNIGKDTCWKLVGIHTCINLDVRESCFMGTLDQWGIGVGRKKWKSSFQHHHLRKKDKDFSSMRTNIGMPGRM

SEQ ID NO: 21 - DNA, 564 bp, Artificial Sequence
  Chemically Synthesized, Human IL-36 gamma full length
atggattggacctggattctgtttctggtcgccgctgctacaagagtgcattcaagaggaacccccggagatgctgatggaggaggacgggccgtgtaccagagcatgtgcaagcctatcaccggcacaatcaacgacctgaatcagcaagtgtggaccctgcagggacagaacctggtggcagtgccaaggtctgatagcgtgacccccgtgacagtggccgtgatcacctgtaagtatccagaggccctggagcagggacgcggcgaccctatctacctgggcatccagaatccagagatgtgcctgtattgtgagaaagtgggcgagcagccaaccctgcagctgaaggagcagaagatcatggatctgtacggccagcccgagcctgtgaagcccttcctgttttatcgggccaagacaggcagaacctccacactggagtctgtggccttccccgactggtttatcgccagctccaagagggatcagcctatcatcctgacctctgaactggggaaatcctacaatactgcttttgaactgaacattaacgactgataa

SEQ ID NO: 22 - protein, 186 aa, Artificial Sequence
  Chemically Synthesized, Human IL-36 gamma full length
MDWTWILFLVAAATRVHSRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 23 - DNA, 516 bp, Artificial Sequence
  Chemically Synthesized, Human IL-36 gamma truncated
atggactggacctggattctgttcctggtcgctgccgctacaagagtgcattcaagcatgtgcaagcctattactgggactatcaatgacctgaaccagcaagtgtggaccctgcagggccagaatctggtggccgtgccaaggtctgatagcgtgacccccgtgacagtggccgtgatcacatgcaagtaccctgaggccctggagcagggacgcggcgaccctatctacctgggcatccagaacccagagatgtgcctgtattgtgagaaagtgggcgagcagccaaccctgcagctgaaggagcagaagatcatggatctgtacggccagcccgagcctgtgaagcccttcctgttttatcgggccaagacaggcagaacctccacactggagtctgtggccttccccgactggtttatcgccagctccaagcgggatcagcctatcattctgacctccgaactggggaagtcctacaatacagcctttgaactgaacattaacgactgataa

SEQ ID NO: 24 - protein, 170 aa, Artificial Sequence
  Chemically Synthesized, Human IL-36 gamma truncated
MDWTWILFLVAAATRVHSSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 25 - DNA, 537 bp, Artificial Sequence
  Chemically Synthesized, Full length IL-36 alpha
atggactggacctggattctgttcctcgtcgccgccgcaactagggtgcattcaaacaaagaaaaagaactccgggctgcctccccctccctgaggcacgtgcaggacctcagctccagagtctggattctgcagaacaatattctcaccgctgtgcccaggaaggagcagacagtgcctgtcactatcaccctgctcccatgccagtacctggacacactcgaaactaaccggggcgaccccacatatatgggtgtgcagcgccctatgtcttgcctgttctgtactaaggatggagagcagcctgtcctgcagctcggagagggaaacatcatggaaatgtacaataagaaggaaccagtgaaagcaagtctgttctatcacaagaaatcagggaccacaagcacctttgagtccgccgctttcccaggctggtttatcgccgtgtgctctaaaggaagttgtcccctgattctcactcaggagctgggtgaaatcttcattaccgactttgaaatgatcgtggtccattgataa

SEQ ID NO: 26 - protein, 177 aa, Artificial Sequence
  Chemically Synthesized, Full length IL-36 alpha
MDWTWILFLVAAATRVHSNKEKELRAASPSLRHVQDLSSRVWILQNNILTAVPRKEQTVPVTITLLPCQYLDTLETNRGDPTYMGVQRPMSCLFCTKDGEQPVLQLGEGNIMEMYNKKEPVKASLFYHKKSGTTSTFESAAFPGWFIAVCSKGSCPLILTQELGEIFITDFEMIVVH

SEQ ID NO: 27 - DNA, 519 bp, Artificial Sequence
  Chemically Synthesized, Truncated IL-36 alpha
atggactggacttggattctcttcctggtcgcagccgctacacgggtgcattcacgagccgcttccccttcactgaggcatgtgcaggacctgagcagcagggtgtggattctgcagaacaatattctcaccgctgtccccaggaaggagcagacagtgcctgtcactatcaccctgctcccatgccagtacctggacacactcgaaactaacaggggcgaccccacttatatgggtgtgcagagacctatgtcttgcctgttctgtaccaaggatggagagcagcctgtcctgcagctcggagagggaaacatcatggaaatgtacaataagaaggaaccagtgaaagcaagcctcttctatcacaagaaatcaggaaccacaagcacatttgagtccgccgctttcccaggctggtttattgccgtgtgctctaaaggaagttgtcccctgattctgacccaggaactgggggagattttcattactgattttgagatgattgtcgtccattgataa

SEQ ID NO: 28 - protein, 171 aa, Artificial Sequence
  Chemically Synthesized, Truncated IL-36 alpha
MDWTWILFLVAAATRVHSRAASPSLRHVQDLSSRVWILQNNILTAVPRKEQTVPVTITLLPCQYLDTLETNRGDPTYMGVQRPMSCLFCTKDGEQPVLQLGEGNIMEMYNKKEPVKASLFYHKKSGTTSTFESAAFPGWFIAVCSKGSCPLILTQELGEIFITDFEMIVVH

SEQ ID NO: 29 - DNA, 603 bp, Artificial Sequence
  Chemically Synthesized, Full length IL-36 beta
atggattggacctggattctgttcctcgtcgccgccgcaactagagtgcattctgccttcccccctcagtcatgtgtgcatgtgctgccccctaagagcatccagatgtgggagcctaaccacaataccatgcatgggagctcccagtccccaaggaactacagagtgcacgactctcagcagatggtgtgggtcctgacaggtaataccctcacagccgtgccagctagtaacaatgtgaaacccgtcatcctgtcactcattgcttgccgagacactgaattccaggatgtgaagaaagggaacctggtctttctcggtattaagaacagaaatctgtgtttctgctgtgtggagatggaagggaaacccaccctgcagctcaaggaggtcgatatcatgaatctgtacaaggaaaggaaagcacagaaggccttcctgttttatcacggcattgagggatcaacaagcgtgttccagtctgtcctgtaccctggctggtttatcgccacttctagtattgaacggcagactatcattctgacccatcagcgcggaaaactcgtgaacacaaacttctacatcgagagcgaaaagtgataa

SEQ ID NO: 30 - protein, 200 aa, Artificial Sequence
  Chemically Synthesized, Full length IL-36 beta
MDWTWILFLVAAATRVHSMAFPPQSCVHVLPPKSIQMWEPNHNTMHGSSQSPRNYRVHDSQQMVWVLTGNTLTAVPASNNVKPVILSLIACRDTEFQDVKKGNLVFLGIKNRNLCFCCVEMEGKPTLQLKEVDIMNLYKERKAQKAFLFYHGIEGSTSVFQSVLYPGWFIATSSIERQTIILTHQRGKLVNTNFYIESEK

SEQ ID NO: 31 - DNA, 519 bp, Artificial Sequence
  Chemically Synthesized, Truncated IL-36 beta
atggactggacttggattctcttcctggtcgccgcagcaacacgagtgcattcctcctcacagagcccccgcaactatcgagtgcatgactcccagcagatggtgtgggtcctgacaggcaacaccctcacagccgtgcccgcttcaaacaatgtgaaacctgtcatcctgagcctcattgcttgcagggacactgagttccaggatgtgaagaaagggaatctggtctttctcggtattaagaacagaaacctgtgcttctgctgcgtggagatggaaggcaaaccaaccctgcagctcaaggaggtggatattatgaacctgtacaaggaacggaaagcacagaaggccttcctcttttatcacggcatcgagggcagcaccagcgtgttccagagtgtcctgtaccccggatggtttatcgccactagctccattgaaaggcagaccatcatcctgacccaccagagaggcaaactcgtgaatacaaacttctacattgaaagcgaaaaatgataa

SEQ ID NO: 32 - protein, 171 aa, Artificial Sequence
  Chemically Synthesized, Truncated IL-36 beta
MDWTWILFLVAAATRVHSSSQSPRNYRVHDSQQMVWVLTGNTLTAVPASNNVKPVILSLIACRDTEFQDVKKGNLVFLGIKNRNLCFCCVEMEGKPTLQLKEVDIMNLYKERKAQKAFLFYHGIEGSTSVFQSVLYPGWFIATSSIERQTIILTHQRGKLVNTNFYIESEK

SEQ ID NO: 33 - DNA, 636 bp, Artificial Sequence
  Chemically Synthesized, Full length IL-36 gamma
atggactggacatggattctgttcctggtcgctgctgctacacgggtgcattctgaaaataatgaaaagaaaaatatcgtctacggctctgacgtggagatggaacacgagcgagcaggactgttcgtcagctccgctatgttcagtaagcaccccttttcaacccatatcagcggccgggaaacacctgatttcggagaggtgtttgacctcgatcagcaggtctggatttttcgcaatcaggctctggtgacagtccctaggagtcacagagtgacaccagtgtcagtcactatcctcccatgcaagtaccccgaatctctggagcaggacaaagggatcgccatctacctgggtattcagaatcctgataagtgcctgttctgtaaagaagtgaacgggcatccaaccctgctcctgaaggaggaaaaaatcctcgacctgtaccaccatcccgaacctatgaagcccttcctgttttatcacaccagaacaggcggaacttccaccttcgagtctgtggcatttccagggcattacatcgcctctagtaagactggtaaccccattttcctcaccagcaagaaaggagagtactacaacatcaacttcaacctggatatcaagtcctgataa

SEQ ID NO: 34 - protein, 181 aa, Artificial Sequence
  Chemically Synthesized, Full length IL-36 gamma
MDWTWILFLVAAATRVHSFSKHPFSTHISGRETPDFGEVFDLDQQVWIFRNQALVTVPRSHRVTPVSVTILPCKYPESLEQDKGIAIYLGIQNPDKCLFCKEVNGHPTLLLKEEKILDLYHHPEPMKPFLFYHTRTGGTSTFESVAFPGHYIASSKTGNPIFLTSKKGEYYNINFNLDIKS

SEQ ID NO: 35 - DNA, 516 bp, Artificial Sequence
  Chemically Synthesized, Truncated IL-36 gamma
atggattggacatggattctgtttctggtcgccgccgctacaagagtgcatagtggacgggagacacctgatttcggggaagtgtttgacctcgatcagcaagtgtggatcttccggaaccaggcactggtgaccgtccctaggtcacacagagtcacaccagtgagcgtcactatcctcccatgcaagtaccccgagtctctggaacaggacaaagggatcgccatctacctgggtattcagaaccctgataagtgcctgttttgtaaagaagtgaatggccatcccaccctgctcctgaaggaggaaaaaattctcgacctgtaccaccatcccgagcctatgaagcccttcctgttttatcacaccaggacaggcggaacttctaccttcgaaagtgtggcctttcccggccattacatcgctagctccaaaacaggaaatcctattttcctgacaagcaaaaagggagaatactacaacattaacttcaacctggacatcaaatcttgataa

SEQ ID NO: 36 - protein, 170 aa, Artificial Sequence
  Chemically Synthesized, Truncated IL-36 gamma
MDWTWILFLVAAATRVHSGRETPDFGEVFDLDQQVWIFRNQALVTVPRSHRVTPVSVTILPCKYPESLEQDKGIAIYLGIQNPDKCLFCKEVNGHPTLLLKEEKILDLYHHPEPMKPFLFYHTRTGGTSTFESVAFPGHYIASSKTGNPIFLTSKKGEYYNINFNLDIKS

SEQ ID NO: 37 - protein, 18 aa, Artificial Sequence
  Chemically Synthesized, IgE leader sequence
MDWTWILFLVAAATRVHS

(19) United States
(12) Patent Application Publication
(10) Pub. No.: US 2022/0211845 A1
(43) Pub. Date: Jul. 7, 2022

(54) DNA ENCODED IL-36 GAMMA AS AN ADJUVANT

(71) Applicant: THE WISTAR INSTITUTE OF ANATOMY AND BIOLOGY, Philadelphia, PA (US)

(72) Inventors: David B. Weiner, Merion, PA (US); Lumena Louis, Brooklyn, NY (US)

(21) Appl. No.: 17/609,451

(22) PCT Filed: May 8, 2020
(86) PCT No.: PCT/US20/32031

§ 371 Date: Nov. 8, 2021

Related U.S. Application Data
(60) Provisional application No. 62/845,011, filed on May 8, 2019.

(51) Int. Cl. A61K 39/39; A61K 39/21; A61K 39/145; A61K 39/12; A61P 31/16; A61P 31/14; A61P 31/18

(52) U.S. Cl. CPC A61K 39/39; A61K 39/21; A61K 39/145; A61K 2039/53; A61P 31/16; A61P 31/14; A61P 31/18; A61K 39/12

(57) ABSTRACT

Disclosed herein are immunogenic compositions comprising an antigen and an IL-36 adjuvant. Also disclosed herein are methods for increasing an immune response in a subject. The methods may comprise administering the immunogenic compositions to the subject in need thereof.

### CROSS-REFERENCE TO RELATED APPLICATIONS

This application claims priority to U.S. Provisional Application No. 62/845,011, filed May 8, 2019, which is hereby incorporated by reference herein in its entirety.

### TECHNICAL FIELD

The present invention relates to the use of DNA encoded IL-36 gamma as an adjuvant for vaccines, and methods of administering such vaccines.

### BACKGROUND

The most successful approaches to controlling infectious diseases on a global scale have been through vaccination. Vaccines have led to control, eradication or near eradication of several infectious diseases, positively impacting both human longevity and the quality of life. However, much work remains in this area. For many targets, current studies have suggested the need for adjuvants, which can provide a number of benefits including improved vaccine effectiveness. The best adjuvants can boost overall immune responses to a specific vaccine, thereby requiring either a lower dose or fewer immunizations, improving protection and compliance as well as increasing the global vaccine supply for a particular product (Reed et al., 2013, Nat Med, 19:1597-608). Adjuvants can also help skew and tailor the immune response, which may be useful in scenarios where specific correlates of protection are understood. Furthermore, adjuvants can boost immunity and shorten time to induce a protective vaccine response in populations that traditionally have a difficult time mounting protective responses, including the elderly and immunocompromised patients. Adjuvants function through a number of avenues, including antigen depot formation, enhanced antigen uptake and presentation in some cases through polymerization, as well as induction of multiple innate immune systems such as activation of PAMPS or DAMPS. Alum, the most widely used adjuvant among current licensed vaccines, is well documented to enhance humoral immunity (Wen and Shi, 2016, Emerg Microbes Infect, 5:e25-e25; Brito et al., 2013, Semin Immunol, 25:130-45). Newer vaccine adjuvants including MF59, an oil-in-water emulsion system with squalene, monophosphoryl lipid A (MPL) and the Adjuvant Systems group 03 and 04 (AS03, AS04) which are comprised of alum, oil-in-water emulsions, and TLR-4 agonists have also been licensed and shown to improve antibody responses to antigens as well as provide dose sparing among other benefits for humoral responses. However, there is still a major need in the clinic for adjuvants that can improve CTL responses (Harandi, 2018, Semin Immunol, 39:30-34). A particularly active area of focus has been to identify adjuvants that can also boost cellular CD8 T cell immunity. This work includes nontraditional adjuvants such as pathogen-recognition receptor agonists, liposomes, nanoparticles, and gene encoded adjuvants that can potentially jumpstart the innate immune system and work in concert with the adaptive immune arm to drive lasting memory against antigen (Shah et al., 2017, Vaccine Adjuv:1-13). Clinical studies have reported that the addition of plasmid IL-12 as part of an HIV synthetic DNA vaccine combination alone induced T cell response rates similar to combination vaccine prime boost studies using DNA prime and viral vectors (Kalams et al., 2018, J Infect Dis, 208:818-29). This data encourages further investigation of additional less well-studied cytokines as DNA or other potential adjuvants to further broaden immunity and improve cellular as well as humoral immunity for DNA encoded antigens.

The IL-36 family is made up of pro-inflammatory mediators alpha, beta, gamma, as well as antagonist IL-36Ra (Gresnigt and van de Veerdonk, 2013, Semin Immunol, 25:458-65; Clavel et al., 2013, Joint Bone Spine, 80:449-53). This relatively novel cytokine family remains poorly understood, although recent important studies have begun to shed light on their mechanism of action. The IL-36 family is a part of the IL-1 superfamily, of which alpha, beta, and gamma are agonists. Upon binding to the IL-36 receptor IL-36R, and recruitment of the co-receptor accessory protein IL-1RAcP, these cytokines activate the NF-κB, MAPK pathway resulting in the stimulation of pro-inflammatory intracellular responses, whereas binding of the antagonist IL-36Ra prevents recruitment of IL-1RAcP and does not lead to intracellular response. IL-36R is primarily expressed on naïve CD4 T cells, but is also found on dendritic cells, while the cytokines are expressed mainly in skin keratinocytes and epithelium, although they are also expressed at low levels in the lung, kidneys, and intestine (Gresnigt and van de Veerdonk, 2013, Semin Immunol, 25:458-65; Dinarello, 2013, Semin Immunol, 25:389-93; Dietrich et al., 2016, Cytokine, 84:88-98; Yazdi and Ghoreschi, 2016, Adv Exp Med Biol, 941:21-29).

Identification of novel molecular adjuvants which can boost and enhance vaccine-mediated immunity and provide dose sparing potential against complex infectious diseases and for immunotherapy of cancer is likely to play a critical role in the next generation of vaccines. Given the number of challenging targets for which no or only partial vaccine options exist, adjuvants that can address some of these concerns are in high demand.

Adjuvants have the potential to boost and broaden immune responses in populations that traditionally have difficulty generating protective responses, including the elderly and immunocompromised people. Shingrix, the latest vaccine approved to protect against reactivation of herpes zoster and postherpetic neuralgia (shingles), is a recombinant vaccine made of glycoprotein E and ASO1 adjuvant, a mixture of both MPL and QS-21, a saponin (Bharucha et al., 2017, Hum Vaccines Immunother, 13:1789-97; Sly and Harris, 2018, Nurs Womens Health, 22:417-22; Ragupathi et al., 2011, Expert Rev Vaccines, 10:463-70). This vaccine demonstrated an efficacy of over 95% against herpes zoster, compared to the efficacy of ZostaVax, a live attenuated vaccine, which is 51% overall (Bharucha et al., 2017, Hum Vaccines Immunother, 13:1789-97; Sly and Harris, 2018, Nurs Womens Health, 22:417-22). The ability of a recombinant protein vaccine to generate more effective immunity than a live attenuated vaccine is in large part due to the effect of the adjuvant included in the formulation. This success has bolstered the adjuvant field, and has reinforced the need for more focus on developing new adjuvants that work in a variety of settings for other vaccine platforms and generating their adjuvant effects through unique mechanisms of action.

Accordingly, a need remains in the art for the development of safe and more effective adjuvants that increase antigenic responses irrespective of the identity of the antigen and route of administration.

### SUMMARY OF THE INVENTION

In one embodiment, the invention relates to an immunogenic composition comprising a nucleic acid molecule encoding an antigen and a nucleic acid molecule encoding an optimized IL-36 adjuvant. In one embodiment, the IL-36 adjuvant is optIL-36α, optIL-36β, optIL-36γ, a fragment of optIL-36α, a fragment of optIL-36β, a fragment of optIL-36γ, a variant of optIL-36α, a variant of optIL-36β, or a variant of optIL-36γ.

In one embodiment, the nucleic acid molecule encoding an optimized IL-36 adjuvant encodes SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In one embodiment, the nucleic acid molecule encoding an optimized IL-36 adjuvant encodes a variant having at least 95% identity to SEQ ID NO:2, SEQ ID NO:4, SEQ ID NO:6, SEQ ID NO:8, SEQ ID NO:10, SEQ ID NO: 12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In one embodiment, the nucleic acid molecule encoding an optimized IL-36 adjuvant encodes a fragment of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36 comprising at least 60% of the full length sequence. In one embodiment, the nucleic acid molecule encoding an optimized IL-36 adjuvant encodes a variant having at least 95% identity to a fragment of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36, wherein the fragment comprises at least 60% of the full length sequence.

In one embodiment, the nucleic acid molecule encoding an optimized IL-36 adjuvant comprises a nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. In one embodiment, the nucleic acid molecule encoding an optimized IL-36 adjuvant comprises a nucleotide having at least 95% identity to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. In one embodiment, the nucleic acid molecule encoding an optimized IL-36 adjuvant comprises a fragment of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35 comprising at least 60% of the full length sequence. In one embodiment, the nucleic acid molecule encoding an optimized IL-36 adjuvant comprises a variant having at least 95% identity to a fragment of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35, wherein the fragment comprises at least 60% of the full length sequence.

In one embodiment, the antigen is an HIV antigen, an influenza antigen, a ZIKA virus (ZIKV) antigen or a fragment thereof.

In one embodiment, the HIV antigen is Env A, Env B, Env C, Env D, B Nef-Rev, Gag, or any combination thereof.

In one embodiment, the influenza antigen is H1 HA, H2 HA, H3 HA, H5 HA, BHA antigen or any combination thereof.

In one embodiment, the ZIKV antigen includes a prME antigen.

In one embodiment, the composition further comprises a pharmaceutically acceptable excipient.

In one embodiment, the nucleic acid molecule encoding an antigen and the nucleic acid molecule encoding an optimized IL-36 adjuvant are expression vectors.

In one embodiment, the invention relates to a method for increasing an immune response in a subject, the method comprising administering the immunogenic composition comprising a nucleic acid molecule encoding an antigen and a nucleic acid molecule encoding an optimized IL-36 adjuvant.

In one embodiment, the method of administering the immunogenic composition is intramuscular administration or intradermal administration.

In one embodiment, the method of administering the immunogenic composition includes electroporation.

In one embodiment, the increased immune response occurs in at least one of a skin tissue and a muscle tissue of the subject.

In one embodiment, the immune response in the subject is increased by about 75% to about 200%.

In one embodiment, the immune response in the subject is increased by at least about 1.5 fold.

In one embodiment, the invention relates to a nucleic acid molecule comprising one or more nucleotide sequences encoding an optimized IL-36 adjuvant. In one embodiment, the IL-36 adjuvant is optIL-36α, optIL-36β, optIL-36γ, a fragment of optIL-36α, a fragment of optIL-36β, a fragment of optIL-36γ, a variant of optIL-36α, a variant of optIL-36β, or a variant of optIL-36γ.

In one embodiment, the nucleic acid molecule encoding an optimized IL-36 adjuvant encodes SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In one embodiment, the nucleic acid molecule encoding an optimized IL-36 adjuvant encodes a variant having at least 95% identity to SEQ ID NO:2, SEQ ID NO:4, SEQ ID NO:6, SEQ ID NO:8, SEQ ID NO:10, SEQ ID NO: 12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In one embodiment, the nucleic acid molecule encoding an optimized IL-36 adjuvant encodes a fragment of SEQ ID NO:2, SEQ ID NO:4, SEQ ID NO: 6, SEQ ID NO: 8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36 comprising at least 60% of the full length sequence. In one embodiment, the nucleic acid molecule encoding an optimized IL-36 adjuvant encodes a variant having at least 95% identity to a fragment of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36, wherein the fragment comprises at least 60% of the full length sequence.

In one embodiment, the nucleic acid molecule encoding an optimized IL-36 adjuvant comprises a nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. In one embodiment, the nucleic acid molecule encoding an optimized IL-36 adjuvant comprises a nucleotide having at least 95% identity to SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. In one embodiment, the nucleic acid molecule encoding an optimized IL-36 adjuvant comprises a fragment of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35 comprising at least 60% of the full length sequence. In one embodiment, the nucleic acid molecule encoding an optimized IL-36 adjuvant comprises a variant having at least 95% identity to a fragment of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35, wherein the fragment comprises at least 60% of the full length sequence.

In one embodiment, the nucleic acid molecule is a plasmid.

### BRIEF DESCRIPTION OF THE DRAWINGS

FIG. 1A through FIG. 1D depict exemplary experimental results demonstrating that truncation of IL-36 beta enhances immune responses to HIV Env DNA vaccine. FIG. 1A depicts a map of plasmid construct design. Full length IL-36 beta plasmid and IL-36 beta truncated 9 amino acids N-terminal to anchoring residue. Each construct contains a CMV promoter followed by an IgE leader sequence (MDWTWILFLVAAATRVHS; SEQ ID NO:37). FIG. 1B depicts the immunization delivery schedule. B6 mice were immunized three times at three week intervals. FIG. 1C depicts env specific CD4 and CD8 T cell responses by intracellular cytokine staining after peptide stimulation. FIG. 1D depicts the opt-36βt dosing curve of Env specific CD4 and CD8 T cell responses by intracellular cytokine staining after peptide stimulation.

FIG. 2A through FIG. 2C depict exemplary experimental results demonstraing expression of truncated IL-36 constructs. FIG. 2A depicts a map of plasmid construct design for IL-36 sequences. Each sequence was truncated 9 amino acids N-terminal to conserved A-X-Asp residue. Each construct contains a CMV promoter followed by an IgE leader sequence beside the IL-36 sequence. FIG. 2B depicts U2OS cells were transfected with each truncated IL-36 plasmids that contained a C-terminal HA tag for detection. Lysates from these cells were used in Western blot for detection of plasmid expression. FIG. 2C depicts that immunofluorescence analysis was performed on HEK-293T cells transfected with truncated IL-36 plasmids to verify plasmid expression.

FIG. 3A through FIG. 3C depict exemplary experimental results demonstrating that co-delivery of truncated IL-36 beta and gamma enhance immune responses against HIV Env DNA vaccine. FIG. 3A depicts the immunization delivery schedule. B6 mice were immunized three times 3 weeks apart with Env alone or Env adjuvanted with the opt-36αt, opt-36βt, or opt-36γt. Sera and spleens were harvested 50 days post final vaccination to analyze antigen specific immune responses. FIG. 3B depicts the frequency of Env specific IFN gamma responses (spot forming units per million splenocytes) induced after vaccination was determined by IFN gamma ELISpot assay in response to pooled Env peptides. FIG. 3C depicts Env specific CD4 and CD8 T cell responses by intracellular cytokine staining after peptide stimulation.

FIG. 4A and FIG. 4B depict exemplary experimental results demonstrating the humoral response induced post vaccination. FIG. 4A depicts an ELISA analysis measuring binding antibody production (measured by OD450 values) in immunized mice. The C57BL/6 mice (n=5) were immunized intramuscularly three times three weeks apart with 2.5 μg of HIV Env plasmid or 2.5 μg of Env plasmid and 11 μg of opt-36αt, opt-36βt, or opt-36γt. Binding to consensus C gp120 was analyzed with sera from animals post final vaccination. FIG. 4B depicts the average endpoint titers.

FIG. 5A through FIG. 5D depicts exemplary experimental results demonstrating codelivery of truncated IL-36 gamma enhances binding antibody while maintaining antibody integrity. FIG. 5A depicts the immunization delivery schedule. Balb/C mice were immunized two times two weeks apart with influenza H1 alone or H1 adjuvanted with opt-36αt, opt-36βt, or opt36γt. Sera and spleens were harvested two weeks post final vaccination to analyze antigen specific responses. FIG. 5B depicts the frequency of HA specific IFN gamma responses (spot forming units per million splenocytes) induced after vaccination was determined by IFN gamma ELISpot assay in response to pooled HA peptides. FIG. 5C depicts endpoint binding titers post vaccination with H1 alone or H1+ truncated IL-36 adjuvant. FIG. 5D depicts the avidity of antibodies generated after vaccination at 1:50 dilution.

FIG. 6A and FIG. 6B depict exemplary experimental results demonstrating that there was no observed isotype switching. FIG. 6A depicts an ELISA analysis measuring isotype binding antibody production (measured by OD 450 values) in immunized mice. Balb/C (n=4-5) were immunized twice two weeks apart with 1 μg of H1 DNA plasmid or H1 DNA plasmid and 11 μg of opt-36αt, opt-36βt, or opt-36γt. Isotypes of antibodies generated were analyzed with sera from animals post final vaccination. FIG. 6B depicts the IgG₂ₐ/IgG₁ antibody ratio, which was analyzed by dividing the OD 450 values of IgG₂ₐ by the OD 450 values of IgG₁.

FIG. 7A through FIG. 7C depict exemplary experimental results demonstrating co-delivery of truncated IL-36 gamma enhances immune response to DNA pRME vaccine. FIG. 7A depicts the immunization schedule for Zika vaccine immunization. IFNAR−/− mice were immunized once either vaccine alone or vaccine+opt-36γt (n=5-6 per group). Spleens were harvested two weeks post vaccination to analyze antigen specific T cell responses. FIG. 7B depicts the frequency of spot forming units per million splenocytes determined by IFN gamma ELISpot assay in response to pooled Zika pRME peptides. FIG. 7C depicts Zika pRME specific CD4 and CD8 T cell responses by intracellular cytokine staining.

FIG. 8 depicts exemplary experimental results demonstrating induction of ZIKV specific cellular immune responses following vaccination with either ZIKV-prME DNA vaccine alone or opt-36γt alone. ELISpot analysis measuring IFN-γ secretion in splenocytes after one immunization.

FIG. 9A through FIG. 9D depicts exemplary experimental results demonstrating that truncated IL-36 gamma is able to protect against Zika challenge induced weight loss and mortality. FIG. 9A depicts the immunization delivery schedule. IFNAR−/− mice were immunized with Zika prME plasmid or pRME+opt-36γt once and challenged with Zika PR209 virus two weeks later. FIG. 9B depicts that mouse body weight was tracked over the two week challenge period. FIG. 9C depicts the clinical symptoms of immunized mice days 5-7 post challenge. FIG. 9D depicts survival curves of mice post Zika challenge over 14 days.

### DETAILED DESCRIPTION

The present invention relates to vaccines that can be used to increase an immune response to an antigen in a subject by using IL-36, or a fragment thereof, as an adjuvant. IL-36, or a fragment thereof, can safely direct inflammatory responses in multiple tissues such as skin, muscle, etc.

In some instances, IL-36, or a fragment thereof, can function as a universal adjuvant because a greater immune response is elicited in the subject regardless of the source of the antigen or the route of administration as compared to a vaccine comprising the antigen alone. IL-36, or a fragment thereof, may further augment the immune response of both viral and cancer antigens. In some instances, IL-36, or a fragment thereof, can further augment the immune response in both muscle and skin tissues as demonstrated by increased interferon-γ (IFN-γ) production.

The vaccines of the present invention can also unexpectedly modify or alter epitope presentation to increase the immune response to the antigen. Such modification can be dependent upon IL-36, or a fragment thereof. In some instances, IL-36, or a fragment thereof, can direct the immune system to recognize new epitopes in the antigen, in addition to the epitopes recognized by the immune system in the absence of IL-36. In other instances, IL-36 can remap the landscape of epitope recognition by the immune system to increase the immune response to the antigen across tissues and irrespective of the antigen's identity or source.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Adjuvant” as used herein means any molecule added to the vaccines described herein to enhance the immunogenicity of the antigens.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered.

“Complement” or “complementary” as used herein means Watson-Crick (e.g., A-T/U and C-G) or Hoogsteen base pairing between nucleotides or nucleotide analogs of nucleic acid molecules.

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Fragment” or “immunogenic fragment” as used herein means a nucleic acid sequence or a portion thereof that encodes a polypeptide capable of eliciting and/or increasing an immune response in a mammal. The fragments can be DNA fragments selected from at least one of the various nucleotide sequences that encode protein fragments set forth below. Fragments can comprise at least 10%, at least 20%, at least 30%, at least 40%, at least 50%, at least 60%, at least 70%, at least 80%, at least 90%, or at least 95% of one or more of the nucleic acid sequences set forth below. In some embodiments, fragments can comprise at least 20 nucleotides or more, at least 30 nucleotides or more, at least 40 nucleotides or more, at least 50 nucleotides or more, at least 60 nucleotides or more, at least 70 nucleotides or more, at least 80 nucleotides or more, at least 90 nucleotides or more, at least 100 nucleotides or more, at least 150 nucleotides or more, at least 200 nucleotides or more, at least 250 nucleotides or more, at least 300 nucleotides or more, at least 350 nucleotides or more, at least 400 nucleotides or more, at least 450 nucleotides or more, at least 500 nucleotides or more, at least 550 nucleotides or more, at least 600 nucleotides or more, at least 650 nucleotides or more, at least 700 nucleotides or more, at least 750 nucleotides or more, at least 800 nucleotides or more, at least 850 nucleotides or more, at least 900 nucleotides or more, at least 950 nucleotides or more, or at least 1000 nucleotides or more of at least one of the nucleic acid sequences set forth below.

Fragment or immunogenic fragment as used herein also means a polypeptide sequence or a portion thereof that is capable of eliciting and/or increasing an immune response in a mammal. The fragments can be polypeptide fragments selected from at least one of the various amino acid sequences set forth below. Fragments can comprise at least 10%, at least 20%, at least 30%, at least 40%, at least 50%, at least 60%, at least 70%, at least 80%, at least 90%, or at least 95% of one or more of the proteins set forth below. In some embodiments, fragments can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, or at least 240 amino acids or more of at least one of the proteins set forth below.

“Genetic construct” or “construct” as used herein refers to the DNA or RNA molecules that comprise a nucleotide sequence which encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs or constructs that contain the necessary regulatory elements operably linked to a coding sequence that encodes a protein such that when present in the cell of the individual, the coding sequence will be expressed.

“Identical” or “identity” as used herein in the context of two or more nucleic acid or polypeptide sequences means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of the single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

“Immune response” as used herein means the activation of a host's immune system, e.g., that of a mammal, in response to the introduction of an antigen. The immune response can be in the form of a cellular or humoral immune response, or both.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene from which the promoter is derived. As is known in the art, variation in this distance can be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Signal peptide” and “leader sequence” are used interchangeably herein and refer to an amino acid sequence that can be linked at the amino terminus of a protein or amino acid sequence set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the amino terminus of the protein.

“Subject” as used herein can mean a mammal that wants to or is in need of being immunized with the herein described vaccines. The mammal can be a human, chimpanzee, dog, cat, horse, cow, mouse, or rat.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides, or that the two sequences hybridize under stringent hybridization conditions.

“Substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more amino acids. Substantially identical can also mean that a first nucleic acid sequence and a second nucleic acid sequence are at least 60%, 65%, 70%, 75%, 80%, 81%, 82%, 83%, 84%, 85%, 86%, 87%, 88%, 89%, 90%, 9l %, 92%, 93%, 94%, 95%, 96%, 97%, 98%, or 99% identical over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides.

“Treatment” or “treating” as used herein can mean protecting an animal from a disease through means of preventing, suppressing, repressing, or completely eliminating the disease. Preventing the disease involves administering a vaccine of the present invention to an animal prior to onset of the disease. Suppressing the disease involves administering a vaccine of the present invention to an animal after induction of the disease but before its clinical appearance. Repressing the disease involves administering a vaccine of the present invention to an animal after clinical appearance of the disease.

“Variant” as used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof, or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid. The vector can contain or include one or more heterologous nucleic acid sequences.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. Vaccines

Provided herein are immunogenic compositions (e.g., vaccines) comprising an antigen and an adjuvant. The immunogenic compositions can increase antigen presentation and the overall immune response to the antigen in an individual. The combination of antigen and adjuvant induces the immune system more efficiently than a vaccine comprising the antigen alone. The immunogenic compositions can further modify epitope presentation within the antigen to induce a greater immune response to the antigen than immunogenic compositions comprising the antigen alone. The immunogenic compositions can further induce an immune response when administered to different tissues such as the muscle and the skin.

The immunogenic composition can be a DNA vaccine, an RNA vaccine, a peptide vaccine, or a combination vaccine. The immunogenic composition of the present invention can have features required of effective vaccines such as being safe so that the immunogenic composition itself does not cause illness or death; being protective against illness resulting from exposure to live pathogens such as viruses or bacteria; inducing neutralizing antibody to prevent infection of cells; inducing protective T cell responses against intracellular pathogens; and providing ease of administration, few side effects, biological stability, and low cost per dose. The immunogenic composition can accomplish some or all of these features by combining the antigen with the adjuvant as discussed below.

a. Adjuvant

The immunogenic composition can comprise an adjuvant and antigen as discussed below. The adjuvant can be a nucleic acid sequence, an amino acid sequence, or a combination thereof. The nucleic acid sequence can be DNA, RNA, cDNA, a variant thereof, a fragment thereof, or a combination thereof. The nucleic acid sequence can also include additional sequences that encode linker or tag sequences that are linked to the adjuvant by a peptide bond. The amino acid sequence can be a protein, a peptide, a variant thereof, a fragment thereof, or a combination thereof.

(1) IL-36

The adjuvant can be a member of the interleukin (IL)-36 subfamily of interleukin proteins. IL-36 family proteins that can serve as adjuvants include, but are not limited to, IL-36α, IL-36β and IL-36γ, and fragments and variants thereof, or the combination thereof IL-36 cytokines can direct both innate and adaptive immune responses by acting on parenchymal, stromal, and specific immune cell subsets.

In one embodiment, inclusion of an IL-36 adjuvant in an immunogenic composition or vaccine can induce IFN-γ production by at least about 1.5-fold, at least about 2-fold, at least about 3-fold, at least about 4-fold, at least about 5-fold, at least about 8-fold, and at least about 10-fold as compared to a vaccine not including an IL-36 adjuvant. Inclusion of an IL-36 adjuvant in the vaccine can induce IFN-γ production by at least about 2-fold as compared to a vaccine not including an IL-36 adjuvant. Inclusion of an IL-36 adjuvant in the vaccine can induce IFN-γ production by at least about 3-fold as compared to a vaccine not including an IL-36 adjuvant.

IL-36 can increase or boost the immune response to the antigen in a subject. The antigen is described in more detail below. In some instances, IL-36 can increase the immune response to the antigen by about 75% to about 200%. Alternatively, IL-36 can increase the immune response to the antigen by about 90% to about 130%. In still other alternative embodiments, IL-36 can increase the immune response to the antigen by about 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129% or 130%.

In other embodiments, IL-36 can increase or boost the immune response to the antigen by at least about 1.5-fold, at least about 2-fold, at least about 2.5-fold, at least about 3-fold, at least about 4-fold, at least about 5-fold, at least about 6-fold, at least about 7-fold, at least about 8-fold, at least about 9-fold, or at least about 10-fold when the herein described vaccines are administered to a subject in need thereof.

In some embodiments, IL-36 can modify or alter immune system recognition of at least one epitope in the antigen in any number of tissues in the individual, for example, a skin tissue and a muscle tissue. The antigen is described in more detail below. Such altered recognition of the at least one epitope can induce a greater immune response in a subject administered the herein described immunogenic compositions as compared to a subject administered an immunogenic composition comprising a nucleic acid corresponding to the antigen alone.

IL-36 may also modify or change the presentation of one or more epitopes in the antigen, for example, by allowing a previously unrecognized epitope to be recognized by the immune system, thereby increasing the immune response in the subject to the antigen. The modified presentation, and thus the increased immune response, can occur in any number of tissues in the subject, for example, a skin tissue and a muscle tissue.

A nucleic acid encoding the IL-36 adjuvant can be from any number of organisms, for example, mouse (Mus musculus), macaque (Macacac mulatta), and human (Homo sapiens). The nucleic acid encoding the IL-36 adjuvant can be optimized with regards to codon usage and corresponding RNA transcripts. The nucleic acid encoding the IL-36 adjuvant can be codon and RNA optimized for expression. In some embodiments, the nucleic acid encoding the IL-36 adjuvant can include a Kozak sequence (e.g., GCC ACC) to increase the efficiency of translation. The nucleic acid encoding the IL-36 adjuvant can include multiple stop codons (e.g., TGA TGA) to increase the efficiency of translation termination. The nucleic acid encoding the IL-36 adjuvant can also include a nucleotide sequence encoding a IgE leader sequence. The IgE leader sequence can be located 5′ to the IL-36 adjuvant in the nucleic acid. In some embodiments, the nucleic acid encoding the IL-36 adjuvant is free of or does not contain a nucleotide sequence encoding the IgE leader sequence.

In various embodiments, the IL-36 protein included in the adjuvant is an optimized IL-36α, IL-36β, or IL-36γ protein, or a variant or fragment thereof. In one embodiment, the fragments is a truncated fragment of IL-36α, IL-36β, or IL-36γ that lacks 9 amino acids on the N-terminus of the sequence.

TABLE 1
Sequences encoding optimized IL-36 proteins

| SEQ ID NO: | Sequence Type | Description |
|---|---|---|
| 1 | Nucleotide | Human IL-36 alpha full length, no IgE |
| 2 | Amino acid | Human IL-36 alpha full length, no IgE |
| 3 | Nucleotide | Human IL-36 alpha truncated, no IgE |
| 4 | Amino acid | Human IL-36 alpha truncated, no IgE |
| 5 | Nucleotide | Human IL-36 beta full length, no IgE |
| 6 | Amino acid | Human IL-36 beta full length, no IgE |
| 7 | Nucleotide | Human IL-36 beta truncated, no IgE |
| 8 | Amino acid | Human IL-36 beta truncated, no IgE |
| 9 | Nucleotide | Human IL-36 gamma full length, no IgE |
| 10 | Amino acid | Human IL-36 gamma full length, no IgE |
| 11 | Nucleotide | Human IL-36 gamma truncated, no IgE |
| 12 | Amino acid | Human IL-36 gamma truncated, no IgE |
| 13 | Nucleotide | Human IL-36 alpha full length, with IgE |
| 14 | Amino acid | Human IL-36 alpha full length, with IgE |
| 15 | Nucleotide | Human IL-36 alpha truncated, with IgE |
| 16 | Amino acid | Human IL-36 alpha truncated, with IgE |
| 17 | Nucleotide | Human IL-36 beta full length, with IgE |
| 18 | Amino acid | Human IL-36 beta full length, with IgE |
| 19 | Nucleotide | Human IL-36 beta truncated, with IgE |
| 20 | Amino acid | Human IL-36 beta truncated, with IgE |
| 21 | Nucleotide | Human IL-36 gamma full length, with IgE |
| 22 | Amino acid | Human IL-36 gamma full length, with IgE |
| 23 | Nucleotide | Human IL-36 gamma truncated, with IgE |
| 24 | Amino acid | Human IL-36 gamma truncated, with IgE |
| 25 | Nucleotide | Murine IL-36 alpha full length, with IgE |
| 26 | Amino acid | Murine IL-36 alpha full length, with IgE |
| 27 | Nucleotide | Murine IL-36 alpha truncated, with IgE |
| 28 | Amino acid | Murine IL-36 alpha truncated, with IgE |
| 29 | Nucleotide | Murine IL-36 beta full length, with IgE |
| 30 | Amino acid | Murine IL-36 beta full length, with IgE |
| 31 | Nucleotide | Murine IL-36 beta truncated, with IgE |
| 32 | Amino acid | Murine IL-36 beta truncated, with IgE |
| 33 | Nucleotide | Murine IL-36 gamma full length, with IgE |
| 34 | Amino acid | Murine IL-36 gamma full length, with IgE |
| 35 | Nucleotide | Murine IL-36 gamma truncated, with IgE |
| 36 | Amino acid | Murine IL-36 gamma truncated, with IgE |

In one embodiment, the nucleotide sequence encoding an optimized IL-36α (optIL-36α) protein can be the nucleic acid sequence SEQ ID NO: 1, which encodes for SEQ ID NO: 2. In one embodiment, the nucleotide sequence encoding an optIL-36α protein is operably linked to one or more regulatory sequences. In one embodiment, the nucleotide sequence encoding an optIL-36α protein is operably linked to an IgE leader sequence. In one embodiment, the nucleotide sequence encoding an optimized IL-36α (optIL-36α) protein operably linked to an IgE leader sequence can be the nucleic acid sequence SEQ ID NO: 13, which encodes for SEQ ID NO: 14. In one embodiment, the nucleotide sequence encoding an optimized IL-36α (optIL-36α) protein can be optimized for expression in mice. In one embodiment, the nucleotide sequence encoding an optimized IL-36α (optIL-36α) protein operably linked to an IgE leader sequence and optimized for expression in mice can be the nucleic acid sequence SEQ ID NO: 25, which encodes for SEQ ID NO: 26.

In some embodiments, the nucleotide sequence encoding an optIL-36α protein can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence of SEQ ID NO:1, SEQ ID NO:13 or SEQ ID NO:25. In other embodiments, the nucleotide sequence encoding an optIL-36α protein can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:2, SEQ ID NO:14 or SEQ ID NO:26. The optIL-36α protein can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:2, SEQ ID NO:14 or SEQ ID NO:26.

In one embodiment, the nucleotide sequence encoding an optimized truncated IL-36α protein (optIL-36α t) can be the nucleic acid sequence SEQ ID NO: 3, which encodes for SEQ ID NO: 4. In one embodiment, the nucleotide sequence encoding an optIL-36αt protein is operably linked to one or more regulatory sequences. In one embodiment, the nucleotide sequence encoding an optIL-36αt protein is operably linked to an IgE leader sequence. In one embodiment, the nucleotide sequence encoding an optIL-36αt protein operably linked to an IgE leader sequence can be the nucleic acid sequence SEQ ID NO: 15, which encodes for SEQ ID NO: 16. In one embodiment, the nucleotide sequence encoding an optIL-36αt protein can be optimized for expression in mice. In one embodiment, the nucleotide sequence encoding an optIL-36αt protein operably linked to an IgE leader sequence and optimized for expression in mice can be the nucleic acid sequence SEQ ID NO: 27, which encodes for SEQ ID NO: 28.

In some embodiments, the nucleotide sequence encoding an optIL-36αt protein can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence of SEQ ID NO:3, SEQ ID NO:15 or SEQ ID NO:27. In other embodiments, the nucleotide sequence encoding an optIL-36αt protein can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:4, SEQ ID NO: 16 or SEQ ID NO:28. The optIL-36αt protein can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:4, SEQ ID NO:16 or SEQ ID NO:28.

In one embodiment, the nucleotide sequence encoding an optimized IL-36β (optIL-36β) protein can be the nucleic acid sequence SEQ ID NO: 5, which encodes for SEQ ID NO:6. In one embodiment, the nucleotide sequence encoding an optIL-36β protein is operably linked to one or more regulatory sequences. In one embodiment, the nucleotide sequence encoding an optIL-36β protein is operably linked to an IgE leader sequence. In one embodiment, the nucleotide sequence encoding an optIL-36β protein operably linked to an IgE leader sequence can be the nucleic acid sequence SEQ ID NO:17, which encodes for SEQ ID NO:18. In one embodiment, the nucleotide sequence encoding an optIL-36β protein can be optimized for expression in mice. In one embodiment, the nucleotide sequence encoding an optIL-36β protein operably linked to an IgE leader sequence and optimized for expression in mice can be the nucleic acid sequence SEQ ID NO: 29, which encodes for SEQ ID NO:30.

In some embodiments, the nucleotide sequence encoding an optIL-363 protein can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence of SEQ ID NO:5, SEQ ID NO:17 or SEQ ID NO:29. In other embodiments, the nucleotide sequence encoding an optIL-36β protein can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:6, SEQ ID NO:18 or SEQ ID NO:30. The optIL-36β protein can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:6, SEQ ID NO:18 or SEQ ID NO:30.

In one embodiment, the nucleotide sequence encoding an optimized truncated IL-36β protein (optIL-36βt) can be the nucleic acid sequence SEQ ID NO: 7, which encodes for SEQ ID NO: 8. In one embodiment, the nucleotide sequence encoding an optIL-36βt protein is operably linked to one or more regulatory sequences. In one embodiment, the nucleotide sequence encoding an optIL-36βt protein is operably linked to an IgE leader sequence. In one embodiment, the nucleotide sequence encoding an optIL-36βt protein operably linked to an IgE leader sequence can be the nucleic acid sequence SEQ ID NO:19, which encodes for SEQ ID NO:20. In one embodiment, the nucleotide sequence encoding an optIL-36βt protein can be optimized for expression in mice. In one embodiment, the nucleotide sequence encoding an optIL-36βt protein operably linked to an IgE leader sequence and optimized for expression in mice can be the nucleic acid sequence SEQ ID NO: 31, which encodes for SEQ ID NO:32.

In some embodiments, the nucleotide sequence encoding an optIL-36βt protein can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence of SEQ ID NO:7, SEQ ID NO:19 or SEQ ID NO:31. In other embodiments, the nucleotide sequence encoding an optIL-36βt protein can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:8, SEQ ID NO:20 or SEQ ID NO:32. The optIL-36βt protein can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:8, SEQ ID NO:20 or SEQ ID NO:32.

In one embodiment, the nucleotide sequence encoding an optimized IL-36γ (optIL-36γ) protein can be the nucleic acid sequence SEQ ID NO: 9, which encodes for SEQ ID NO: 10. In one embodiment, the nucleotide sequence encoding an optIL-36γ protein is operably linked to one or more regulatory sequences. In one embodiment, the nucleotide sequence encoding an optIL-36γ protein is operably linked to an IgE leader sequence. In one embodiment, the nucleotide sequence encoding an optIL-36γ protein operably linked to an IgE leader sequence can be the nucleic acid sequence SEQ ID NO:21, which encodes for SEQ ID NO:22. In one embodiment, the nucleotide sequence encoding an optIL-36γ protein can be optimized for expression in mice. In one embodiment, the nucleotide sequence encoding an optIL-36γ protein operably linked to an IgE leader sequence and optimized for expression in mice can be the nucleic acid sequence SEQ ID NO: 33, which encodes for SEQ ID NO:34.

In some embodiments, the nucleotide sequence encoding an optIL-36γ protein can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence of SEQ ID NO:9, SEQ ID NO:21 or SEQ ID NO:33. In other embodiments, the nucleotide sequence encoding an optIL-36γ protein can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:10, SEQ ID NO:22 or SEQ ID NO:34. The optIL-36γ protein can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:10, SEQ ID NO:22 or SEQ ID NO:34.

In one embodiment, the nucleotide sequence encoding an optimized truncated IL-36γ protein (optIL-36γt) can be the nucleic acid sequence SEQ ID NO: 11, which encodes for SEQ ID NO: 12. In one embodiment, the nucleotide sequence encoding an optIL-36γt protein is operably linked to one or more regulatory sequences. In one embodiment, the nucleotide sequence encoding an optIL-36γt protein is operably linked to an IgE leader sequence. In one embodiment, the nucleotide sequence encoding an optIL-36γt protein operably linked to an IgE leader sequence can be the nucleic acid sequence SEQ ID NO:23, which encodes for SEQ ID NO:24. In one embodiment, the nucleotide sequence encoding an optIL-36γt protein can be optimized for expression in mice. In one embodiment, the nucleotide sequence encoding an optIL-36γt protein operably linked to an IgE leader sequence and optimized for expression in mice can be the nucleic acid sequence SEQ ID NO: 35, which encodes for SEQ ID NO:36.

In some embodiments, the nucleotide sequence encoding an optIL-36γt protein can be the nucleic acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the nucleic acid sequence of SEQ ID NO:11, SEQ ID NO:23 or SEQ ID NO:35. In other embodiments, the nucleotide sequence encoding an optIL-36γt protein can be the nucleic acid sequence that encodes the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:12, SEQ ID NO:24 or SEQ ID NO:36. The optIL-36γt protein can be the amino acid sequence having at least about 80%, 81%, 82%, 83%, 84%, 85%, 86%, 87%, 88%, 89%, 90%, 91%, 92%, 93%, 94%, 95%, 96%, 97%, 98%, 99%, or 100% identity over an entire length of the amino acid sequence of SEQ ID NO:12, SEQ ID NO:24 or SEQ ID NO:36.

Some embodiments relate to fragments of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. Fragments can comprise at least 60%, at least 65%, at least 70%, at least 75%, at least 80%, at least 85%, at least 90%, at least 95%, at least 96%, at least 97%, at least 98%, or at least 99% of the full length sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In some embodiments, fragments can include sequences that encode a leader sequence, for example, an immunoglobulin leader sequence, such as the IgE leader sequence. In some embodiments, fragments are free of coding sequences that encode a leader sequence.

Some embodiments relate to fragments of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. Fragments can comprise at least 60%, at least 65%, at least 70%, at least 75%, at least 80%, at least 85%, at least 90%, at least 95%, at least 96%, at least 97%, at least 98%, or at least 99% of the full length sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. In some embodiments, fragments can include sequences that encode a leader sequence, for example, an immunoglobulin leader sequence, such as the IgE leader sequence. In some embodiments, fragments are free of coding sequences that encode a leader sequence.

Variants of fragments of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35 can be provided. Such variants can comprise fragments comprising at least 60%, at least 65%, at least 70%, at least 75%, at least 80%, at least 85%, at least 90%, at least 95%, at least 96%, at least 97%, at least 98%, or at least 99% of the full length sequencing having 95% or greater identity to SEQ ID NO:1, SEQ ID NO:3, SEQ ID NO:5, SEQ ID NO:7, SEQ ID NO:9, SEQ ID NO:11, SEQ ID NO: 13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. Some embodiments relate to variants that have 96% or greater identity a fragment of IL-36 (i.e., IL-36α, IL-36β, or IL-36γ) nucleic acid sequences herein. Some embodiments relate to variants that have 97% or greater identity to the fragments of IL-36 (i.e., IL-36α, IL-36β, or IL-36γ) nucleic acid sequences herein. Some embodiments relate to variants that have 98% or greater identity to the fragments of IL-36 (i.e., IL-36α, IL-36β, or IL-36γ) nucleic acid sequences herein. Some embodiments relate to variants that have 99% or greater identity to the fragments of IL-36 (i.e., IL-36α, IL-36β, or IL-36γ) nucleic acid sequences herein. In some embodiments, fragments or variants of IL-36 nucleic acid sequences include sequences that encode a leader sequence, for example, an immunoglobulin leader sequence such as the IgE leader sequence. In some embodiments, fragments or variants of IL-36 nucleic acid sequences are free of coding sequences that encode a leader sequence.

Variants of fragments of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36 can be provided. Such variants can comprise amino acid sequences having at least 60%, at least 65%, at least 70%, at least 75%, at least 80%, at least 85%, at least 90%, at least 95%, at least 96%, at least 97%, at least 98%, or at least 99% of the full length protein sequence having 95% or greater identity to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. Some embodiments relate to variants that have 96% or greater identity to the fragments of IL-36 (i.e., IL-36α, IL-36β, or IL-36γ) amino acid sequences herein. Some embodiments relate to variants that have 97% or greater identity to the fragments of IL-36 (i.e., IL-36α, IL-36β, or IL-36γ) amino acid sequences herein. Some embodiments relate to variants that have 98% or greater identity to the fragments of IL-36 (i.e., IL-36α, IL-36β, or IL-36γ) amino acid sequences herein. Some embodiments relate to variants that have 99% or greater identity to the fragments of IL-36 (i.e., IL-36α, IL-36β, or IL-36γ) amino acid sequences herein. In some embodiments, fragments include a leader sequence, for example, an immunoglobulin leader sequence such as the IgE leader sequence. In some embodiments, the fragments are free of a leader sequence.

In some embodiments, the optimized IL-36 adjuvant can be encoded by an RNA that is a transcript from a DNA sequence having at least about 96%, 97%, 98%, 99% or 100% identity over an entire length of the nucleic acid sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, or SEQ ID NO:35. In some embodiments, the optimized IL-36 adjuvant can be encoded by an RNA that encodes an amino acid sequence having at least about 96%, 97%, 98%, 99% or 100% identity over an entire length of the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36.

The optimized IL-35 adjuvant can be a peptide having the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In some embodiments, the adjuvant can have an amino acid sequence having at least about 96%, 97%, 98%, 99%, or 100% identity over an entire length of the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. In some embodiments, the adjuvant can be a fragment comprising at least 60%, at least 65%, at least 70%, at least 75%, at least 80%, at least 85%, at least 90%, at least 95%, at least 96%, at least 97%, at least 98%, or at least 99% of the full length sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, or SEQ ID NO:36. Some embodiments relate to variants that have 96% or greater identity to the fragments of IL-36 (i.e., IL-36α, IL-36β, or IL-36γ) amino acid sequences herein. Some embodiments relate to variants that have 97% or greater identity to the fragments of IL-36 (i.e., IL-36α, IL-36β, or IL-36γ) amino acid sequences herein. Some embodiments relate to variants that have 98% or greater identity to the fragments of IL-36 (i.e., IL-36α, IL-36β, or IL-36γ) amino acid sequences herein. Some embodiments relate to variants that have 99% or greater identity to the fragments of IL-36 (i.e., IL-36α, IL-36β, or IL-36γ) amino acid sequences herein. In some embodiments, fragments include a leader sequence, for example, an immunoglobulin leader sequence such as the IgE leader sequence. In some embodiments, the fragments are free of a leader sequence.

b. Antigen

The immunogenic composition can comprise an antigen or fragment or variant thereof and an adjuvant as discussed above. The antigen can be anything that induces an immune response in a subject. Purified antigens are not usually strongly immunogenic on their own and are therefore combined with the adjuvant as described above. The immune response induced by the antigen can be boosted or increased when combined with the adjuvant. Such an immune response can be a humoral immune response and/or a cellular immune response. In some embodiments, the combination of the adjuvant and the antigen can boost or increase a cellular immune response in the subject. In other embodiments, the combination of the adjuvant and the antigen can boost or increase a humoral immune response in the subject.

The antigen can be a nucleic acid sequence, an amino acid sequence, or a combination thereof. The nucleic acid sequence can be DNA, RNA, cDNA, a variant thereof, a fragment thereof, or a combination thereof. The nucleic acid sequence can also include additional sequences that encode linker or tag sequences that are linked to the antigen by a peptide bond. The amino acid sequence can be a protein, a peptide, a variant thereof, a fragment thereof, or a combination thereof.

The antigen can be contained in a protein, a nucleic acid, or a fragment thereof, or a variant thereof, or a combination thereof from any number of organisms, for example, a virus, a parasite, a bacterium, a fungus, or a mammal. The antigen can be associated with an autoimmune disease, allergy, or asthma. In other embodiments, the antigen can be associated with cancer, herpes, influenza, hepatitis B, hepatitis C, human papilloma virus (HPV), or human immunodeficiency virus (HIV). As discussed below, the antigen of the immunogenic composition can be selected from a group consisting of a human papilloma virus (HPV) antigen, an HIV antigen, an influenza antigen, a Plasmodium falciparum antigen and a fragment thereof. The HPV antigen can be selected from the group consisting of HPV16 E6 antigen, an HPV16 E7 antigen and a combination thereof. The HIV antigen can be selected from the group consisting of Env A, Env B, Env C, Env D, B Nef-Rev, Gag, and any combination thereof. The influenza antigen can be selected from the group consisting of H1 HA, H2 HA, H3 HA, H5 HA, BHA antigen and any combination thereof. The Plasmodium falciparum antigen may include a circumsporozoite (CS) antigen.

Some antigens can induce a strong immune response. Other antigens can induce a weak immune response. The antigen can elicit a greater immune response when combined with the adjuvant as described above.

(1) Viral Antigens

The antigen can be a viral antigen, or fragment thereof, or variant thereof. The viral antigen can be from a virus from one of the following families: Adenoviridae, Arenaviridae, Bunyaviridae, Caliciviridae, Coronaviridae, Filoviridae, Hepadnaviridae, Herpesviridae, Orthomyxoviridae, Papovaviridae, Paramyxoviridae, Parvoviridae, Picornaviridae, Poxviridae, Reoviridae, Retroviridae, Rhabdoviridae, or Togaviridae. The viral antigen can be from papilloma viruses, for example, human papillomoa virus (HPV), human immunodeficiency virus (HIV), polio virus, hepatitis B virus, hepatitis C virus, smallpox virus (Variola major and minor), vaccinia virus, influenza virus, rhinoviruses, dengue fever virus, equine encephalitis viruses, rubella virus, yellow fever virus, Norwalk virus, hepatitis A virus, human T-cell leukemia virus (HTLV-I), hairy cell leukemia virus (HTLV-II), California encephalitis virus, Hanta virus (hemorrhagic fever), rabies virus, Ebola fever virus, Marburg virus, Zika virus, measles virus, mumps virus, respiratory syncytial virus (RSV), herpes simplex 1 (oral herpes), herpes simplex 2 (genital herpes), herpes zoster (varicella-zoster, a.k.a., chickenpox), cytomegalovirus (CMV), for example human CMV, Epstein-Barr virus (EBV), flavivirus, foot and mouth disease virus, chikungunya virus, lassa virus, arenavirus, or cancer causing virus.

(a) Hepatitis Antigen

The IL-36 adjuvant of the invention can be associated or combined with a hepatitis virus antigen (i.e., hepatitis antigen), or fragment thereof, or variant thereof. The hepatitis antigen can be an antigen or immunogen from hepatitis A virus (HAV), hepatitis B virus (HBV), hepatitis C virus (HCV), hepatitis D virus (HDV), and/or hepatitis E virus (HEV). In some embodiments, the hepatitis antigen can be a heterologous nucleic acid molecule(s), such as a plasmid(s), which encodes one or more of the antigens from HAV, HBV, HCV, HDV, and HEV. The hepatitis antigen can be full-length or immunogenic fragments of full-length proteins.

The hepatitis antigen can comprise consensus sequences and/or one or more modifications for improved expression. Genetic modifications, including codon optimization, RNA optimization, and the addition of a highly efficient immunoglobulin leader sequence to increase the immunogenicity of the constructs, can be included in the modified consensus sequences. The consensus hepatitis antigen may comprise a signal peptide such as an immunoglobulin signal peptide such as an IgE or IgG signal peptide, and in some embodiments, may comprise an HA tag. The immunogens can be designed to elicit stronger and broader cellular immune responses than corresponding codon optimized immunogens.

The hepatitis antigen can be an antigen from HAV. The hepatitis antigen can be a HAV capsid protein, a HAV non-structural protein, a fragment thereof, a variant thereof, or a combination thereof.

The hepatitis antigen can be an antigen from HCV. The hepatitis antigen can be a HCV nucleocapsid protein (i.e., core protein), a HCV envelope protein (e.g., E1 and E2), a HCV non-structural protein (e.g., NS1, NS2, NS3, NS4a, NS4b, NS5a, and NS5b), a fragment thereof, a variant thereof, or a combination thereof.

The hepatitis antigen can be an antigen from HDV. The hepatitis antigen can be a HDV delta antigen, fragment thereof, or variant thereof.

The hepatitis antigen can be an antigen from HEV. The hepatitis antigen can be a HEV capsid protein, fragment thereof, or variant thereof.

The hepatitis antigen can be an antigen from HBV. The hepatitis antigen can be a HBV core protein, a HBV surface protein, a HBV DNA polymerase, a HBV protein encoded by gene X, fragment thereof, variant thereof, or combination thereof. The hepatitis antigen can be a HBV genotype A core protein, a HBV genotype B core protein, a HBV genotype C core protein, a HBV genotype D core protein, a HBV genotype E core protein, a HBV genotype F core protein, a HBV genotype G core protein, a HBV genotype H core protein, a HBV genotype A surface protein, a HBV genotype B surface protein, a HBV genotype C surface protein, a HBV genotype D surface protein, a HBV genotype E surface protein, a HBV genotype F surface protein, a HBV genotype G surface protein, a HBV genotype H surface protein, fragment thereof, variant thereof, or combination thereof. The hepatitis antigen can be a consensus HBV core protein, or a consensus HBV surface protein.

In some embodiments, the hepatitis antigen can be a HBV genotype A consensus core DNA sequence construct, an IgE leader sequence linked to a consensus sequence for HBV genotype A core protein, or a HBV genotype A consensus core protein sequence.

In other embodiments, the hepatitis antigen can be a HBV genotype B consensus core DNA sequence construct, an IgE leader sequence linked to a consensus sequence for HBV genotype B core protein, or a HBV genotype B consensus core protein sequence.

In still other embodiments, the hepatitis antigen can be a HBV genotype C consensus core DNA sequence construct, an IgE leader sequence linked to a consensus sequence for HBV genotype C core protein, or a HBV genotype C consensus core protein sequence.

In some embodiments, the hepatitis antigen can be a HBV genotype D consensus core DNA sequence construct, an IgE leader sequence linked to a consensus sequence for HBV genotype D core protein, or a HBV genotype D consensus core protein sequence.

In other embodiments, the hepatitis antigen can be a HBV genotype E consensus core DNA sequence construct, an IgE leader sequence linked to a consensus sequence for HBV genotype E core protein, or a HBV genotype E consensus core protein sequence.

In some embodiments, the hepatitis antigen can be a HBV genotype F consensus core DNA sequence construct, an IgE leader sequence linked to a consensus sequence for HBV genotype F core protein, or a HBV genotype F consensus core protein sequence.

In other embodiments, the hepatitis antigen can be a HBV genotype G consensus core DNA sequence construct, an IgE leader sequence linked to a consensus sequence for HBV genotype G core protein, or a HBV genotype G consensus core protein sequence.

In some embodiments, the hepatitis antigen can be a HBV genotype H consensus core DNA sequence construct, an IgE leader sequence linked to a consensus sequence for HBV genotype H core protein, or a HBV genotype H consensus core protein sequence.

In still other embodiments, the hepatitis antigen can be a HBV genotype A consensus surface DNA sequence construct, an IgE leader sequence linked to a consensus sequence for HBV genotype A surface protein, or a HBV genotype A consensus surface protein sequence.

In some embodiments, the hepatitis antigen can be a HBV genotype B consensus surface DNA sequence construct, an IgE leader sequence linked to a consensus sequence for HBV genotype B surface protein, or a HBV genotype B consensus surface protein sequence.

In other embodiments, the hepatitis antigen can be a HBV genotype C consensus surface DNA sequence construct, an IgE leader sequence linked to a consensus sequence for HBV genotype C surface protein, or a HBV genotype C consensus surface protein sequence.

In still other embodiments, the hepatitis antigen can be a HBV genotype D consensus surface DNA sequence construct, an IgE leader sequence linked to a consensus sequence for HBV genotype D surface protein, or a HBV genotype D consensus surface protein sequence.

In some embodiments, the hepatitis antigen can be a HBV genotype E consensus surface DNA sequence construct, an IgE leader sequence linked to a consensus sequence for HBV genotype E surface protein, or a HBV genotype E consensus surface protein sequence.

In other embodiments, the hepatitis antigen can be a HBV genotype F consensus surface DNA sequence construct, an IgE leader sequence linked to a consensus sequence for HBV genotype F surface protein, or a HBV genotype F consensus surface protein sequence.

In still other embodiments, the hepatitis antigen can be a HBV genotype G consensus surface DNA sequence construct, an IgE leader sequence linked to a consensus sequence for HBV genotype G surface protein, or a HBV genotype G consensus surface protein sequence.

In other embodiments, the hepatitis antigen can be a HBV genotype H consensus surface DNA sequence construct, an IgE leader sequence linked to a consensus sequence for HBV genotype H surface protein, or a HBV genotype H consensus surface protein sequence.

(b) Human Papilloma Virus (HPV) Antigen

The IL-36 adjuvant of the invention can be associated or combined with a human papilloma virus (HPV) antigen, or fragment thereof, or variant thereof. The HPV antigen can be from HPV types 16, 18, 31, 33, 35, 45, 52, and 58, which cause cervical cancer, rectal cancer, and/or other cancers. The HPV antigen can be from HPV types 6 and 11, which cause genital warts, and are known to be causes of head and neck cancer.

The HPV antigens can be the HPV E6 or E7 domains from each HPV type. For example, for HPV type 16 (HPV16), the HPV16 antigen can include the HPV16 E6 antigen, the HPV16 E7 antigen, fragments, variants, or combinations thereof. Similarly, the HPV antigen can be HPV 6 E6 and/or E7, HPV 11 E6 and/or E7, HPV 18 E6 and/or E7, HPV 31 E6 and/or E7, HPV 33 E6 and/or E7, HPV 52 E6 and/or E7, or HPV 58 E6 and/or E7, fragments, variants, or combinations thereof.

(c) RSV Antigen

The IL-36 adjuvant of the invention can also be associated or combined with an RSV antigen or fragment thereof, or variant thereof. The RSV antigen can be a human RSV fusion protein (also referred to herein as “RSV F”, “RSV F protein” and “F protein”), or fragment or variant thereof. The human RSV fusion protein can be conserved between RSV subtypes A and B. The RSV antigen can be a RSV F protein, or fragment or variant thereof, from the RSV Long strain (GenBank AAX23994.1). The RSV antigen can be a RSV F protein from the RSV A2 strain (GenBank AAB59858.1), or a fragment or variant thereof. The RSV antigen can be a monomer, a dimer or trimer of the RSV F protein, or a fragment or variant thereof. The RSV antigen can be consensus RSV F amino acid sequence, or fragment or variant thereof. The RSV antigen can be an optimized nucleic acid encoding RSV F amino acid sequence or fragment or variant thereof.

The postfusion form of RSV F elicits high titer neutralizing antibodies in immunized animals and protects the animals from RSV challenge. The present invention utilizes this immunoresponse in the claimed immunogenic compositions. According to the invention, the RSV F protein can be in a prefusion form or a postfusion form.

The RSV antigen can also be human RSV attachment glycoprotein (also referred to herein as “RSV G”, “RSV G protein” and “G protein”), or fragment or variant thereof. The human RSV G protein differs between RSV subtypes A and B. The antigen can be RSV G protein, or fragment or variant thereof, from the RSV Long strain (GenBank AAX23993). The RSV antigen can be RSV G protein from: the RSV subtype B isolate H5601, the RSV subtype B isolate H1068, the RSV subtype B isolate H5598, the RSV subtype B isolate H1123, or a fragment or variant thereof. The RSV antigen can be a consensus RSV G amino acid sequence, or fragment or variant thereof. The RSV antigen can be an optimized nucleic acid encoding RSV G amino acid sequence or fragment or variant thereof.

In other embodiments, the RSV antigen can be human RSV non-structural protein 1 (“NS1 protein”), or fragment or variant thereof. For example, the RSV antigen can be RSV NS1 protein, or fragment or variant thereof, from the RSV Long strain (GenBank AAX23987.1). The RSV antigen human can also be RSV non-structural protein 2 (“NS2 protein”), or fragment or variant thereof. For example, the RSV antigen can be RSV NS2 protein, or fragment or variant thereof, from the RSV Long strain (GenBank AAX23988.1). The RSV antigen can further be human RSV nucleocapsid (“N”) protein, or fragment or variant thereof. For example, the RSV antigen can be RSV N protein, or fragment or variant thereof, from the RSV Long strain (GenBank AAX23989.1). The RSV antigen can be human RSV Phosphoprotein (“P”) protein, or fragment or variant thereof. For example, the RSV antigen can be RSV P protein, or fragment or variant thereof, from the RSV Long strain (GenBank AAX23990.1). The RSV antigen also can be human RSV Matrix protein (“M”) protein, or fragment or variant thereof. For example, the RSV antigen can be RSV M protein, or fragment or variant thereof, from the RSV Long strain (GenBank AAX23991.1).

In still other embodiments, the RSV antigen can be human RSV small hydrophobic (“SH”) protein, or fragment or variant thereof. For example, the RSV antigen can be RSV SH protein, or fragment or variant thereof, from the RSV Long strain (GenBank AAX23992.1). The RSV antigen can also be human RSV Matrix protein2-1 (“M2-1”) protein, or fragment or variant thereof. For example, the RSV antigen can be RSV M2-1 protein, or fragment or variant thereof, from the RSV Long strain (GenBank AAX23995.1). The RSV antigen can further be human RSV Matrix protein 2-2 (“M2-2”) protein, or fragment or variant thereof. For example, the RSV antigen can be RSV M2-2 protein, or fragment or variant thereof, from the RSV Long strain (GenBank AAX23997.1). The RSV antigen human can be RSV Polymerase L (“L”) protein, or fragment or variant thereof. For example, the RSV antigen can be RSV L protein, or fragment or variant thereof, from the RSV Long strain (GenBank AAX23996.1).

In further embodiments, the RSV antigen can have a consensus amino acid sequence of NS1, NS2, N, P, M, SH, M2-1, M2-2, or L protein. The RSV antigen can be a human RSV protein or recombinant antigen, such as any one of the proteins encoded by the human RSV genome.

In other embodiments, the RSV antigen can be, but is not limited to, the RSV F protein from the RSV Long strain, the RSV G protein from the RSV Long strain, the consensus RSV G amino acid sequence, the optimized nucleic acid encoding RSV G amino acid sequence, the human RSV genome of the RSV Long strain, the consensus RSV F amino acid sequence, the optimized nucleic acid encoding RSV F amino acid sequence, the RSV NS1 protein from the RSV Long strain, the RSV NS2 protein from the RSV Long strain, the RSV N protein from the RSV Long strain, the RSV P protein from the RSV Long strain, the RSV M protein from the RSV Long strain, the RSV SH protein from the RSV Long strain, the RSV M2-1 protein from the RSV Long strain, for the RSV M2-2 protein from the RSV Long strain, the RSV L protein from the RSV Long strain, the RSV G protein from the RSV subtype B isolate H5601, the RSV G protein from the RSV subtype B isolate H1068, for the RSV G protein from the RSV subtype B isolate H5598, the RSV G protein from the RSV subtype B isolate H1123, or fragment thereof, or variant thereof.

(d) Influenza Antigen

The IL-36 adjuvant of the invention can be associated or combined with an influenza antigen or fragment thereof, or variant thereof. The influenza antigens are those capable of eliciting an immune response in a mammal against one or more influenza serotypes. The antigen can comprise the full length translation product HA0, subunit HA1, subunit HA2, a variant thereof, a fragment thereof or a combination thereof. The influenza hemagglutinin antigen can be a consensus sequence derived from multiple strains of influenza A serotype H1, a consensus sequence derived from multiple strains of influenza A serotype H2, a hybrid sequence containing portions of two different consensus sequences derived from different sets of multiple strains of influenza A serotype H1 or a consensus sequence derived from multiple strains of influenza B. The influenza hemagglutinin antigen can be from influenza B.

The influenza antigen can also contain at least one antigenic epitope that can be effective against particular influenza immunogens against which an immune response can be induced. The antigen may provide an entire repertoire of immunogenic sites and epitopes present in an intact influenza virus. The antigen may be a consensus hemagglutinin antigen sequence that can be derived from hemagglutinin antigen sequences from a plurality of influenza A virus strains of one serotype such as a plurality of influenza A virus strains of serotype H1 or of serotype H2. The antigen may be a hybrid consensus hemagglutinin antigen sequence that can be derived from combining two different consensus hemagglutinin antigen sequences or portions thereof. Each of two different consensus hemagglutinin antigen sequences may be derived from a different set of a plurality of influenza A virus strains of one serotype such as a plurality of influenza A virus strains of serotype H1. The antigen may be a consensus hemagglutinin antigen sequence that can be derived from hemagglutinin antigen sequences from a plurality of influenza B virus strains.

In some embodiments, the influenza antigen can be H1 HA, H2 HA, H3 HA, H5 HA, or a BHA antigen. Alternatively, the influenza antigen can be a consensus hemagglutinin antigen comprising a consensus H1 amino acid sequence or a consensus H2 amino acid sequence. The consensus hemagglutinin antigen may be a synthetic hybrid consensus H1 sequence comprising portions of two different consensus H1 sequences, which are each derived from a different set of sequences from the other. An example of a consensus HA antigen that is a synthetic hybrid consensus H1 protein is a protein comprising the U2 amino acid sequence. The consensus hemagglutinin antigen may be a consensus hemagglutinin protein derived from hemagglutinin sequences from influenza B strains, such as a protein comprising the consensus BHA amino acid sequence.

The consensus hemagglutinin antigen may further comprise one or more additional amino acid sequence elements. The consensus hemagglutinin antigen may further comprise on its N-terminus, an IgE or IgG leader amino acid sequence. The consensus hemagglutinin antigen may further comprise an immunogenic tag, which is a unique immunogenic epitope that can be detected by readily available antibodies. An example of such an immunogenic tag is the 9 amino acid influenza HA Tag, which may be linked on the consensus hemagglutinin C-terminus. In some embodiments, consensus hemagglutinin antigen may further comprise on its N-terminus, an IgE or IgG leader amino acid sequence and on its C-terminus, an HA tag.

The consensus hemagglutinin antigen may be a consensus hemagglutinin protein that consists of consensus influenza amino acid sequences or fragments and variants thereof. The consensus hemagglutinin antigen may be a consensus hemagglutinin protein that comprises non-influenza protein sequences and influenza protein sequences or fragments and variants thereof.

Examples of a consensus H1 protein include those that may consist of the consensus H1 amino acid sequence or those that further comprise additional elements such as an IgE leader sequence, or an HA Tag or both an IgE leader sequence and an HA Tag.

Examples of consensus H2 proteins include those that may consist of the consensus H2 amino acid sequence or those that further comprise an IgE leader sequence, or an HA Tag, or both an IgE leader sequence and an HA Tag.

Examples of hybrid consensus H1 proteins include those that may consist of the consensus U2 amino acid sequence or those that further comprise an IgE leader sequence, or an HA Tag, or both an IgE leader sequence and an HA Tag.

Examples of hybrid consensus influenza B hemagglutinin proteins include those that may consist of the consensus BHA amino acid sequence or it may comprise an IgE leader sequence, or a an HA Tag, or both an IgE leader sequence and an HA Tag.

The consensus hemagglutinin protein can be encoded by a consensus hemagglutinin nucleic acid, a variant thereof or a fragment thereof. Unlike the consensus hemagglutinin protein which may be a consensus sequence derived from a plurality of different hemagglutinin sequences from different strains and variants, the consensus hemagglutinin nucleic acid refers to a nucleic acid sequence that encodes a consensus protein sequence and the coding sequences used may differ from those used to encode the particular amino acid sequences in the plurality of different hemagglutinin sequences from which the consensus hemagglutinin protein sequence is derived. The consensus nucleic acid sequence may be codon optimized and/or RNA optimized. The consensus hemagglutinin nucleic acid sequence may comprise a Kozak sequence in the 5′ untranslated region. The consensus hemagglutinin nucleic acid sequence may comprise nucleic acid sequences that encode a leader sequence. The coding sequence of an N terminal leader sequence is 5′ of the hemagglutinin coding sequence. The N-terminal leader can facilitate secretion. The N-terminal leader can be an IgE leader or an IgG leader. The consensus hemagglutinin nucleic acid sequence can comprise nucleic acid sequences that encode an immunogenic tag. The immunogenic tag can be on the C-terminus of the protein and the sequence encoding it is 3′ of the consensus HA coding sequence. The immunogenic tag provides a unique epitope for which there are readily available antibodies so that such antibodies can be used in assays to detect and confirm expression of the protein. The immunogenic tag can be an HA Tag at the C-terminus of the protein.

(e) Human Immunodeficiency Virus (HIV) Antigen

The IL-36 adjuvant of the invention can be associated or combined with an HIV antigen or fragment thereof, or variant thereof. HIV antigens can include modified consensus sequences for immunogens. Genetic modifications, including codon optimization, RNA optimization, and the addition of a highly efficient immunoglobin leader sequence to increase the immunogenicity of constructs, can be included in the modified consensus sequences. The novel immunogens can be designed to elicit stronger and broader cellular immune responses than a corresponding codon optimized immunogen.

In some embodiments, the HIV antigen can be a subtype A consensus envelope DNA sequence construct, an IgE leader sequence linked to a consensus sequence for Subtype A envelope protein, or a subtype A consensus Envelope protein sequence.

In other embodiments, the HIV antigen can be a subtype B consensus envelope DNA sequence construct, an IgE leader sequence linked to a consensus sequence for Subtype B envelope protein, or an subtype B consensus Envelope protein sequence

In still other embodiments, the HIV antigen can be a subtype C consensus envelope DNA sequence construct, an IgE leader sequence linked to a consensus sequence for subtype C envelope protein, or a subtype C consensus envelope protein sequence.

In further embodiments, the HIV antigen can be a subtype D consensus envelope DNA sequence construct, an IgE leader sequence linked to a consensus sequence for Subtype D envelope protein, or a subtype D consensus envelope protein sequence.

In some embodiments, the HIV antigen can be a subtype B Nef-Rev consensus envelope DNA sequence construct, an IgE leader sequence linked to a consensus sequence for Subtype B Nef-Rev protein, or a Subtype B Nef-Rev consensus protein sequence

In other embodiments, the HIV antigen can be a Gag consensus DNA sequence of subtype A, B, C and D DNA sequence construct, an IgE leader sequence linked to a consensus sequence for Gag consensus subtype A, B, C and D protein, or a consensus Gag subtype A, B, C and D protein sequence.

In still other embodiments the HIV antigen can be a MPol DNA sequence or a MPol protein sequence. The HIV antigen can be nucleic acid or amino acid sequences of Env A, Env B, Env C, Env D, B Nef-Rev, Gag, or any combination thereof.

(f) ZIKA Virus (ZIKV)/Flaviviridae Family Antigen

The IL-36 adjuvant of the invention can be associated or combined with a Flaviviridae family antigen, or fragment thereof, or variant thereof. The Flaviviridae family genus comprises about 70 different viruses, including human-pathogenic arthropod-borne viruses, such as yellow fever (YF), dengue (Den), West Nile (WN), Japanese encephalitis (JE), tick-borne encephalitis (TBE) viruses and Zika Virus (ZIKV). In one embodiment, the IL-36 adjuvant of the invention can be associated or combined with a ZIKV antigen. The ZIKV antigen can be C, capsid protein; prM, precursor of membrane protein (M); E, envelope protein, fragments, variants, or combinations thereof.

(2) Parasite Antigens

The antigen can be a parasite antigen or fragment or variant thereof. The parasite can be a protozoa, helminth, or ectoparasite. The helminth (i.e., worm) can be a flatworm (e.g., flukes and tapeworms), a thorny-headed worm, or a round worm (e.g., pinworms). The ectoparasite can be lice, fleas, ticks, and mites.

The parasite can be any parasite causing the following diseases: Acanthamoeba keratitis, Amoebiasis, Ascariasis, Babesiosis, Balantidiasis, Baylisascariasis, Chagas disease, Clonorchiasis, Cochliomyia, Cryptosporidiosis, Diphyllobothriasis, Dracunculiasis, Echinococcosis, Elephantiasis, Enterobiasis, Fascioliasis, Fasciolopsiasis, Filariasis, Giardiasis, Gnathostomiasis, Hymenolepiasis, Isosporiasis, Katayama fever, Leishmaniasis, Lyme disease, Malaria, Metagonimiasis, Myiasis, Onchocerciasis, Pediculosis, Scabies, Schistosomiasis, Sleeping sickness, Strongyloidiasis, Taeniasis, Toxocariasis, Toxoplasmosis, Trichinosis, and Trichuriasis.

The parasite can be Acanthamoeba, Anisakis, Ascaris lumbricoides, Botfly, Balantidium coli, Bedbug, Cestoda (tapeworm), Chiggers, Cochliomyia hominivorax, Entamoeba histolytica, Fasciola hepatica, Giardia lamblia, Hookworm, Leishmania, Linguatula serrata, Liver fluke, Loa loa, Paragonimus-lung fluke, Pinworm, Plasmodium falciparum, Schistosoma, Strongyloides stercoralis, Mite, Tapeworm, Toxoplasma gondii, Trypanosoma, Whipworm, or Wuchereria bancrofti.

(a) Malaria Antigen

The IL-36 adjuvant of the invention can be associated or combined with a malaria antigen (i.e., PF antigen or PF immunogen), or fragment thereof, or variant thereof. The antigen can be from a parasite causing malaria. The malaria causing parasite can be Plasmodium falciparum. The Plasmodium falciparum antigen can include the circumsporozoite (CS) antigen.

In some embodiments, the malaria antigen can be nucleic acid molecules such as plasmids which encode one or more of the P. falciparum immunogens CS; LSA1; TRAP; CelTOS; and Amal. The immunogens may be full length or immunogenic fragments of full length proteins. The immunogens can comprise consensus sequences and/or modifications for improved expression.

In other embodiments, the malaria antigen can be a consensus sequence of TRAP, which is also referred to as SSP2, designed from a compilation of all full-length Plasmodium falciparum TRAP/SSP2 sequences in the GenBank database (28 sequences total). Consensus TRAP immunogens (i.e., ConTRAP immunogen) may comprise a signal peptide such as an immunoglobulin signal peptide such as an IgE or IgG signal peptide and in some embodiments, may comprise an HA Tag.

In still other embodiments, the malaria antigen can be CelTOS, which is also referred to as Ag2 and is a highly conserved Plasmodium antigen. Consensus CelTOS antigens (i.e., ConCelTOS immunogen) may comprise a signal peptide such as an immunoglobulin signal peptide such as an IgE or IgG signal peptide and in some embodiments, may comprise an HA Tag.

In further embodiments, the malaria antigen can be Amal, which is a highly conserved Plasmodium antigen. The malaria antigen can also be a consensus sequence of Amal (i.e., ConAmaI immunogen) comprising in some instances, a signal peptide such as an immunoglobulin signal peptide such as an IgE or IgG signal peptide and in some embodiments, may comprise an HA Tag.

In some embodiments, the malaria antigen can be a consensus CS antigen (i.e., Consensus CS immunogen) comprising in some instances, a signal peptide such as an immunoglobulin signal peptide such as an IgE or IgG signal peptide and in some embodiments, may comprise an HA Tag.

In other embodiments, the malaria antigen can be a fusion protein comprising a combination of two or more of the PF proteins set forth herein. For example, fusion proteins may comprise two or more of Consensus CS immunogen, ConLSA1 immunogen, ConTRAP immunogen, ConCelTOS immunogen and ConAmal immunogen linked directly adjacent to each other or linked with a spacer or one more amino acids in between. In some embodiments, the fusion protein comprises two PF immunogens. In some embodiments the fusion protein comprises three PF immunogens. In some embodiments, the fusion protein comprises four PF immunogens. In some embodiments the fusion protein comprises five PF immunogens.

Fusion proteins with two Consensus PF immunogens may comprise: CS and LSA1; CS and TRAP; CS and CelTOS; CS and Amal; LSA1 and TRAP; LSA1 and CelTOS; LSA1 and Amal; TRAP and CelTOS; TRAP and Amal; or CelTOS and Amal. Fusion proteins with three Consensus PF immunogens may comprise: CS, LSA1 and TRAP; CS, LSA1 and CelTOS; CS, LSA1 and Amal; LSA1, TRAP and CelTOS; LSA1, TRAP and Amal; or TRAP, CelTOS and Amal. Fusion proteins with four Consensus PF immunogens may comprise: CS, LSA1, TRAP and CelTOS; CS, LSA1, TRAP and Amal; CS, LSA1, CelTOS and Amal; CS, TRAP, CelTOS and Amal; or LSA1, TRAP, CelTOS and Amal. Fusion proteins with five Consensus PF immunogens may comprise CS or CS-alt, LSA1, TRAP, CelTOS and Amal.

In some embodiments, the fusion proteins comprise a signal peptide linked to the N-terminus. In some embodiments, the fusion proteins comprise multiple signal peptides linked to the N-terminus of each Consensus PF immunogen. In some embodiments, a spacer may be included between PF immunogens of a fusion protein. In some embodiments, the spacer between PF immunogens of a fusion protein may be a proteolyic cleavage site. In some embodiments, the spacer may be a proteolyic cleavage site recognized by a protease found in cells to which the immunogenic composition is intended to be administered and/or taken up. In some embodiments, a spacer may be included between PF immunogens of a fusion protein, wherein the spacer is a proteolyic cleavage site recognized by a protease found in cells to which the immunogenic composition is intended to be administered and/or taken up and the fusion protein comprises multiple signal peptides linked to the N-terminus of each Consensus PF immunogens such that upon cleavage, the signal peptide of each Consensus PF immunogen translocates the respective Consensus PF immunogen to outside the cell.

(3) Bacterial Antigens

The antigen can be bacterial antigen or fragment or variant thereof. The bacterium can be from any one of the following phyla: Acidobacteria, Actinobacteria, Aquificae, Bacteroidetes, Caldiserica, Chlamydiae, Chlorobi, Chloroflexi, Chrysiogenetes, Cyanobacteria, Deferribacteres, Deinococcus-Thermus, Dictyoglomi, Elusimicrobia, Fibrobacteres, Firmicutes, Fusobacteria, Gemmatimonadetes, Lentisphaerae, Nitrospira, Planctomycetes, Proteobacteria, Spirochaetes, Synergistetes, Tenericutes, Thermodesulfobacteria, Thermotogae, and Verrucomicrobia.

The bacterium can be a gram positive bacterium or a gram negative bacterium. The bacterium can be an aerobic bacterium or an anerobic bacterium. The bacterium can be an autotrophic bacterium or a heterotrophic bacterium. The bacterium can be a mesophile, a neutrophile, an extremophile, an acidophile, an alkaliphile, a thermophile, psychrophile, halophile, or an osmophile.

The bacterium can be an anthrax bacterium, an antibiotic resistant bacterium, a disease causing bacterium, a food poisoning bacterium, an infectious bacterium, Salmonella bacterium, Staphylococcus bacterium, Streptococcus bacterium, or tetanus bacterium. The bacterium can be a mycobacteria, Clostridium tetani, Yersinia pestis, Bacillus anthracis, methicillin-resistant Staphylococcus aureus (MRSA), or Clostridium difficile.

(a) Mycobacterium tuberculosis Antigens

The IL-36 adjuvant of the invention can be associated or combined with a Mycobacterium tuberculosis antigen (i.e., TB antigen or TB immunogen), or fragment thereof, or variant thereof. The TB antigen can be from the Ag85 family of TB antigens, for example, Ag85A and Ag85B. The TB antigen can be from the Esx family of TB antigens, for example, EsxA, EsxB, EsxC, EsxD, EsxE, EsxF, EsxH, EsxO, EsxQ, EsxR, EsxS, EsxT, EsxU, EsxV, and EsxW.

In some embodiments, the TB antigen can be heterologous nucleic acid molecules such as plasmids, which encode one or more of the Mycobacterium tuberculosis immunogens from the Ag85 family and the Esx family. The immunogens can be full-length or immunogenic fragments of full-length proteins. The immunogens can comprise consensus sequences and/or modifications for improved expression. Consensus immunogens may comprise a signal peptide such as an immunoglobulin signal peptide such as an IgE or IgG signal peptide and in some embodiments, may comprise an HA tag.

(4) Fungal Antigens

The antigen can be a fungal antigen or fragment or variant thereof. The fungus can be Aspergillus species, Blastomyces dermatitidis, Candida yeasts (e.g., Candida albicans), Coccidioides, Cryptococcus neoformans, Cryptococcus gattii, dermatophyte, Fusarium species, Histoplasma capsulatum, Mucoromycotina, Pneumocystis jirovecii, Sporothrix schenckii, Exserohilum, or Cladosporium.

c. Vector

The immunogenic composition can comprise one or more vectors that include one or more heterologous nucleic acids encoding the antigen and the adjuvant. The one or more vectors can be capable of expressing the antigen and the adjuvant. The one or more vectors can be an expression construct, which is generally a plasmid that is used to introduce a specific gene into a target cell. Once the expression vector is inside the cell, the protein that is encoded by the gene is produced by the cellular-transcription and translation machinery ribosomal complexes. The plasmid is frequently engineered to contain regulatory sequences that act as enhancer and promoter regions and lead to efficient transcription of the gene carried on the expression vector. The vectors of the present invention express large amounts of stable messenger RNA, and therefore proteins.

The vectors may have expression signals such as a strong promoter, a strong termination codon, adjustment of the distance between the promoter and the cloned gene, and the insertion of a transcription termination sequence and a PTIS (portable translation initiation sequence).

(1) Expression Vectors

The vector can be a circular plasmid or a linear nucleic acid. The circular plasmid and linear nucleic acid are capable of directing expression of a particular heterologous nucleotide sequence in an appropriate subject cell. The vector can have a promoter operably linked to the antigen-encoding nucleotide sequence, or the adjuvant-encoding nucleotide sequence, which may be operably linked to termination signals. The vector can also contain sequences required for proper translation of the nucleotide sequence. The vector comprising the nucleotide sequence of interest may be chimeric, meaning that at least one of its components is heterologous with respect to at least one of its other components. The expression of the nucleotide sequence in the expression cassette may be under the control of a constitutive promoter or an inducible promoter, which initiates transcription only when the host cell is exposed to some particular external stimulus. In the case of a multicellular organism, the promoter can also be specific to a particular tissue or organ or stage of development.

(2) RNA Vectors

In one embodiment, the nucleic acid is an RNA molecule. Accordingly, in one embodiment, the invention provides an RNA molecule encoding an optimized IL-36 protein. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

(3) Circular and Linear Vectors

The vector may be circular plasmid, which may transform a target cell by integration into the cellular genome or exist extrachromosomally (e.g. autonomous replicating plasmid with an origin of replication).

The vector can be pVAX, pcDNA3.0, or provax, or any other expression vector capable of expressing heterologous DNA encoding the antigen, or the adjuvant and enabling a cell to translate the sequence to an antigen that is recognized by the immune system, or the adjuvant.

Also provided herein is a linear nucleic acid vaccine, or linear expression cassette (“LEC”), that is capable of being efficiently delivered to a subject via electroporation and expressing one or more desired antigens, and/or one or more desired adjuvants. The LEC may be any linear DNA devoid of any phosphate backbone. The DNA may encode one or more antigens, and/or one or more adjuvants. The LEC may contain a promoter, an intron, a stop codon, and/or a polyadenylation signal. The expression of the antigen, or the adjuvant may be controlled by the promoter. The LEC may not contain any antibiotic resistance genes and/or a phosphate backbone. The LEC may not contain other nucleic acid sequences unrelated to the desired antigen gene expression, or the desired adjuvant expression.

(4) Promoter, Intron, Stop Codon, and Polyadenylation Signal

The vector may have a promoter. A promoter may be any promoter that is capable of driving gene expression and regulating expression of the isolated nucleic acid. Such a promoter is a cis-acting sequence element required for transcription via a DNA dependent RNA polymerase, which transcribes the antigen sequence, or the adjuvant sequence described herein. Selection of the promoter used to direct expression of a heterologous nucleic acid depends on the particular application. The promoter may be positioned about the same distance from the transcription start in the vector as it is from the transcription start site in its natural setting. However, variation in this distance may be accommodated without loss of promoter function.

The promoter may be operably linked to the nucleic acid sequence encoding the antigen and signals required for efficient polyadenylation of the transcript, ribosome binding sites, and translation termination. The promoter may be operably linked to the nucleic acid sequence encoding the adjuvant and signals required for efficient polyadenylation of the transcript, ribosome binding sites, and translation termination.

The promoter may be a CMV promoter, SV40 early promoter, SV40 later promoter, metallothionein promoter, murine mammary tumor virus promoter, Rous sarcoma virus promoter, polyhedrin promoter, or another promoter shown effective for expression in eukaryotic cells.

The vector may include an enhancer and an intron with functional splice donor and acceptor sites. The vector may contain a transcription termination region downstream of the structural gene to provide for efficient termination. The termination region may be obtained from the same gene as the promoter sequence or may be obtained from different genes.

d. Excipients and Other Components of the Immunogenic Composition

The immunogenic composition may further comprise a pharmaceutically acceptable excipient. The pharmaceutically acceptable excipient can be functional molecules such as vehicles, adjuvants other than IL-36, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent can be a polyanion, polycation, including poly-L-glutamate (LGS), or lipid. The transfection facilitating agent can be poly-L-glutamate, and the poly-L-glutamate can be present in the immunogenic composition at a concentration of less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene. Hyaluronic acid can also be used or administered in conjunction with the genetic construct. The DNA plasmid vaccines may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient can be an adjuvant in addition to IL-36. The additional adjuvant can be other genes that are expressed in an alternative plasmid or are delivered as proteins in combination with the plasmid above in the vaccine. The adjuvant may be selected from the group consisting of α-interferon (IFN-α), β-interferon (IFN-β), γ-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes that can be useful as adjuvants in addition to IL-36 include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The immunogenic composition may further comprise a genetic vaccine facilitator agent as described in U.S. Ser. No. 021,579 filed Apr. 1, 1994, which is fully incorporated by reference.

The immunogenic composition can be formulated according to the mode of administration to be used. An injectable vaccine pharmaceutical composition can be sterile, pyrogen free and particulate free. An isotonic formulation or solution can be used. Additives for isotonicity can include sodium chloride, dextrose, mannitol, sorbitol, and lactose. The vaccine can comprise a vasoconstriction agent. The isotonic solutions can include phosphate buffered saline. The immunogenic composition can further comprise stabilizers including gelatin and albumin. The stabilizers can allow the formulation to be stable at room or ambient temperature for extended periods of time, including LGS or polycations or polyanions.

### 3. Methods of Vaccination

The present invention is also directed to methods of increasing an immune response in a subject by different routes of administration of the vaccine. Increasing the immune response can be used to treat and/or prevent disease in the subject.

The method can include administering the herein disclosed vaccines to the subject. The subject administered the vaccine can have an increased or boosted immune response as compared to a subject administered the antigen alone. In some embodiments, the immune response in the subject administered the vaccine can be increased by about 18% to about 650%. Alternatively, the immune response in the subject administered the vaccine may be increased by about 45% to about 260%. In still other alternative embodiments, the immune response in the subject administered the vaccine may be increased by about 93% to about 130%.

In other embodiments, the administered vaccine can increase or boost the immune response in the subject by at least about 1.5-fold, at least about 2-fold, at least about 2.5-fold, at least about 3-fold, at least about 4-fold, at least about 5-fold, at least about 6-fold, at least about 7-fold, at least about 8-fold, at least about 9-fold, or at least about 10-fold.

The vaccine dose can be between 1 μg to 10 mg active component/kg body weight/time, and can be 20 μg to 10 mg component/kg body weight/time. The vaccine can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of vaccine doses for effective treatment can be 1, 2, 3, 4, 5, 6, 7, 8, 9, or 10.

a. Administration

The vaccine can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration. The subject can be a mammal, such as a human, a horse, a cow, a pig, a sheep, a cat, a dog, a rat, or a mouse.

The vaccine can be administered prophylactically or therapeutically. In prophylactic administration, the vaccines can be administered in an amount sufficient to induce an immune response. In therapeutic applications, the vaccines are administered to a subject in need thereof in an amount sufficient to elicit a therapeutic effect. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the vaccine can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The vaccines can be delivered via a variety of routes. Typical delivery routes include parenteral administration, e.g., intradermal, intramuscular or subcutaneous delivery. Other routes include oral administration, intranasal, and intravaginal routes. For the DNA of the vaccine in particular, the vaccine can be delivered to the interstitial spaces of tissues of an individual (Felgner et al., U.S. Pat. Nos. 5,580,859 and 5,703,055, the contents of all of which are incorporated herein by reference in their entirety). The vaccine can also be administered to muscle, or can be administered via intradermal or subcutaneous injections, or transdermally, such as by iontophoresis. Epidermal administration of the vaccine can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can be incorporated into liposomes, microspheres or other polymer matrices (Felgner et al., U.S. Pat. No. 5,703,055; Gregoriadis, Liposome Technology, Vols. I to III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The vaccine can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the vaccine described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the vaccine into tissue without the use of a needle. The MID may inject the vaccine as a small stream or jet with such force that the vaccine pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. Patent Application No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired vaccine in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the vaccine into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver vaccines to all types of tissues, particularly to skin and mucosa. In some embodiments, a needle-free injector may be used to propel a liquid that contains the vaccine to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example, set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle vaccine injectors that deliver the vaccine and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell Pa.) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described vaccine herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired, means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could, for example, be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during electroporation. This is particularly advantageous as it means that the electric field is applied to the same area as the injected fluid. There has traditionally been a problem with electroporation in that it is very difficult to accurately align an electrode with previously injected fluid and so user's have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

The present invention has multiple aspects, illustrated by the following non-limiting examples.

### 3. Examples

### Example 1

Designed DNA Encoded IL-36 Gamma Acts as a Potent Molecular Adjuvant Enhancing Zika Synthetic DNA Vaccine Induced Immunity and Protection in a Lethal Challenge Model

While the IL-36 cytokine family was first discovered nearly two decades ago, it is only recently that roles for these cytokines are beginning to be elucidated. The IL-36 family, members of a larger pro-inflammatory IL-1 family, have been primarily implicated for their potential role in pustular psoriasis and inflammation of the skin and joints (Gresnigt and van de Veerdonk, 2013, Semin Immunol, 25:458-65; Clavel et al., 2013, Joint Bone Spine, 80:449-53; Ding et al., 2017, Oncotarget, 9:2895-901; Foster et al., 2014, J Immunol Baltim Md. 1950; 192:6053-61). Without being bound by theory, it is believed that dysregulation of the natural IL-36 receptor antagonist can help maintain excess activation of the cytokines and mediate epithelial damage when expressed locally. However, some of these same properties have also piqued the scientific community's interest regarding some of the other roles that these cytokines might play. Following reports that IL-36 beta could amplify Th1 responses in CD4+ T cells (Vigne et al., 2012, Blood, 120:3478-87), a number of studies have shown the induction of IL-36 cytokine expression, especially IL-36 gamma, in response to infections including pneumonia, HSV, and candidiasis (Verma et al., 2018, J Immunol, 201:627-34; Winkle et al., Front Microbiol, 7:955; Gardner and Herbst-Kralovetz, 2018, Cytokine, 111:63-71; Kovach et al., 2017, Mucosal Immunol, 10:1320-34; Milora et al., 2017, Sci Rep, 7(1):5799; Aoyagi et al., 2017, PLoS Pathog, 13(11):e1006737), suggesting at the very least that IL-36 cytokines may play a role in host immunity.

The data presented herein compares the effects of three truncated IL-36 cytokines in a vaccination model. These studies demonstrate the ability of truncated IL-36 gamma's (opt-36γt) to boost immune responses using three DNA vaccine models.

It is demonstrated that truncation of the IL-36 cytokines nine amino acids N-terminal to the conserved A-X-Asp motif was critical for their activity to enhance vaccine-induced immune responses. Vaccination of B6 mice with HIV Env vaccine coformulated with opt-36βt (truncated IL-36 beta) significantly increased the number of IFN-γ and TNF-α expressing CD4 T cells compared to mice vaccinated with vaccine and opt-36β (full length IL-36 beta). A similar trend was observed for CD8 T cells.

In the HIV DNA vaccine model, mice immunized with opt-36βt and opt-36γt were both able to enhance vaccine-induced cellular immune responses. However, where opt-36βt was able to significantly increase the number of antigen-specific IFN-γ and TNF-α CD4 T cells, opt-36γt significantly increased the number of antigen-specific IFN-γ, TNF-α, and CD107a+ CD8 T cells, suggesting an impact of opt-36γt to improve cytolytic activity of these antigen specific CD8 T cells. Further work must be done to understand the differences between the two cytokines' seemingly preferential action on various cell compartments. Regarding humoral immunity in this model, opt-36γt was able to increase antibody-binding titers, while opt-36βt appeared to suppress the antibody response, suggesting that opt-36γt may be able to improve both arms of immune response, which likely has importance for many of the challenging disease targets that remain.

Given the potency of the adjuvant effect observed in the HIV vaccine model, the designed cytokines were tested in a dose sparing influenza DNA vaccine model. Mice immunized with vaccine and either opt-36βt or opt-36γt were both able to enhance cellular immune responses as determined by ELISpot assay. Initially, it appeared that mice immunized by opt-36βt were also able to enhance antibody-binding titers against the H1 influenza immunogen, in contrast to what was seen in the HIV Env DNA vaccine model. However, upon further investigation, it was found that the antibodies generated post vaccination with opt-36βt were weaker in affinity for the hemmuglutanin (HA) protein, compared to the antibodies generated post vaccination with opt-36γt, again suggesting opt-36γt's ability to boost both cellular and humoral immunity.

Given the positive data observed in the HIV and influenza DNA vaccine models, opt-36γt and its ability to enhance immune responses was the focus of a non-protective Zika vaccine and challenge model. IFNAR^{-/-} mice that were immunized just once with a very small dose of 500 ng of Zika pRME vaccine or 11 μg of opt-36γt alone were unable to mount an immune response, however the combination of the two resulted in a synergy that generated a robust cellular response similar to that observed when mice were immunized with 25 μg of Zika pRME vaccine alone, highlighting the potential of opt-36γt to reduce necessary vaccine dose. Although the antibodies generated post Zika vaccination were low (but higher than in the absence of opt-36γt), the addition of opt-36γt was still able to protect against Zika challenge, highlighting the importance of cellular immunity in this model. Such an outcome would be important in field vaccination studies where rapid protection might be achieved by a combination of humoral and cellular immunity in a rapid time frame.

There is still much work to be done to fully understand the roles that the IL-36 cytokines play under both homeostatic and pathologic conditions in the host immune system. Understanding how opt-36βt and opt-36γt may exert their activities on different cell populations and against additional vaccine targets in a number of antigenic models will be important for further harnessing their potential.

Given their ability to enhance CD4 and CD8 T cell responses, opt-36γt and opt-36βt look especially promising as potential adjuvants for disease models in which cellular responses are important, such as cancer models where driving CD8 immunity is important to clear tumors. Studies examining the effects of opt-36γt on driving tumor-infiltrating lymphocytes (TILS) would be relevant for example. Work by Wang et al has shown that IL-36γ is able to promote antitumor immune responses in a melanoma model, further strengthening this possibility (Aoyagi et al., 2017, PLoS Pathog, 13(11): e1006737).

The induction of better quality antibodies by opt-36γt as seen in the influenza studies may have a critical role in models in which high affinity antibodies are important. As more emphasis is being focused to identify immunogens that can elicit broadly neutralizing antibodies (bNabs) for HIV and Influenza, adjuvants that can further refine the antibody response may prove important.

Although there appears to be a deleterious effect on skin health when IL-36 signaling is left unchecked, localized delivery of opt-36γt as an adjuvant during intradermal vaccination could enhance protection against infections that breach the skin's natural barrier and recruit immune cells to fight off a number of pathogens that enter the body through the epithelial barrier, including herpes, malaria, and Leishmania among others. As the global population and the demand for vaccines increase worldwide, the need to maximize immune responses while minimizing the effective dose necessary to induce protective responses will continue to grow to further control costs. Opt-36γt, which has shown adjuvant potential in both a dose sparing and non-protective vaccine dose model, may represent a potential avenue to meet some of these demands.

### The Materials and Methods are Now Described

DNA Constructs

The HIV consensus clade C Envelope, Influenza HA, and Zika pRME DNA vaccines used in these studies are as previously described (Yan et al., 2011, Vaccine, 29:7173-81; Scott et al., 2015, Hum Vaccines Immunother, 11:1972-82; Muthumani et al., 2016, Npj Vaccines, 1:16021).

The sequences for murine IL-36 alpha, beta, and gamma were obtained from Uniprot (Q9JLA2-1, Q9D6Z6-1, Q8R460-1). These sequences have been modified to be RNA and codon optimized in order to exploit the host's natural codon preference and enhance protein expression. Furthermore, a highly efficient IgE leader sequence was inserted at the 5′ end of the IL-36 gene to promote efficient secretion of the protein. These full-length optimized IL-36 cytokine plasmids are referred to herein as opt-36α, opt-36β, and opt-36γ.

Recent work by Towne et al (2011, J Biol Chem, 286:42594-602) has demonstrated the need for truncation of IL-36 cytokines nine amino acids N-terminal to a conserved A-X-Asp motif, for full activity. The second set of IL-36 plasmids have been truncated and are henceforth known as opt-36αt, opt-36βt, and opt-36γt. All inserts were modified as previously explained above for enhanced expression and cloned into the pGX0001 backbone (Genscript, Piscataway, N.J.) (Kumar et al., 2006, DNA Cell Biol, 25:383-92).

Western Blot

Transfections were performed using the TurboFectin 8.0 reagent, following the manufacturer's protocols (OnGene, Rockville, Md.). Briefly, HEK 293T cells were grown to 80% confluence in 6 well tissue culture plates and transfected with 2 μg of opt-36αt, opt-36βt, or opt-36γt. The cells were collected 2 days after transfection, washed twice with PBS and lysed with cell lysis buffer (Cell Signaling Technology, Danvers, Mass.). Gradient (4-12%) Bis-Tris NUPAGE gels (Life Technologies, Carlsbad, Calif.) were loaded with transfected cell lysates and transferred to PDVF membrane. The membranes were blocked in PBS Odyssey blocking buffer (LI-COR Biosciences, Lincoln, Nebr., USA) for 1 hour at room temperature. To detect plasmid expression, the anti-HA (A01244 Clone 5E11D8, GenScript) antibody was diluted 1:1000 and anti-R-actin antibody diluted 1:5000 in Odyssey blocking buffer with 0.2% Tween 20 (Bio-Rad, Hercules, Calif.) and incubated with the membranes overnight at 4° C. The membranes were washed with PBST and then incubated with the appropriate secondary antibody (goat anti-mouse IRDye680CW; LI-COR Biosciences) at a 1:15,000 dilution in Odyssey Blocking Buffer for 1 hour at room temperature. After washing, the membranes were imaged on the Odyssey infrared imager (LI-COR Biosciences).

Immunofluorescence Assay (IFA)

For the immunofluorescence assay, HEK 293T cells were grown in 6 well tissue culture slides and transfected with 2 μg of opt-36αt, opt-36βt, or opt-36γt. Two days after transfection, the cells were fixed with 4% paraformaldehyde for 15 minutes. Nonspecific binding was then blocked with normal goat serum diluted in PBS at room temperature for 1 hour. The slides were then washed in PBS for 5 minutes and subsequently incubated with anti-HA antibody at a 1:1000 (mouse anti-HA, GenScript) dilution overnight at 4° C. The slides were washed as described above and incubated with appropriate secondary antibody (goat anti-mouse IgG-AF488, Sigma, St Louis, Mo.) at 1:200 dilutions at room temperature for 1 hour. After washing, DAPI (Millipore Sigma, Burlington, Mass.) was added to stain the nuclei of all cells following manufacturer's protocol. Wells were washed and maintained in PBS, and observed under a microscope (EVOS Cell Imaging Systems; Life Technologies).

Animals

All mice were housed in compliance with the NIH, the University of Pennsylvania School of Medicine and the Wistar Institutional Animal Care and Use Committee (IACUC). Six to eight week old female C57BL/6 mice were purchased from Jackson Laboratory (Bar Harbor, Me.). Five to six week old male and female IFNAR^{-/-} mice were also housed and treated in accordance to the above parties.

Animal Immunizations

For HIV dosing studies, mice were immunized three times at three-week intervals with either 2.5 μg of HIV Env DNA only or 2.5 μg of HIV Env DNA and 11, 20, or 30 μg of opt-36βt in a total volume of 30 μl of water. Mice were injected intramuscularly (IM) in the shaved tibialis anterior muscle followed by electroporation (EP) using the CELLECTRA 3P (Inovio Pharmaceuticals, Plymouth Meeting, Pa.) as previously described (Choi et al., 2019, PLoS Negl Trop Dis, 13:e0007042). For HIV plasmid comparison studies, mice were immunized three times at three-week intervals with either 2.5 μg of HIV Env DNA only or 2.5 μg of HIV Env DNA and 11 μg of opt-36αt, opt-36βt, or opt-36γt in a total volume of 30 μl of water. For influenza studies, mice were immunized two times at two-week intervals with 1 μg of H1 DNA plasmid alone or 1 μg of H1 DNA plasmid and 11 μg of opt-36αt, opt-36βt, or opt-36γt in a total volume of 30 μl of water delivered intramuscularly as described above. For Zika studies, mice were immunized once with 500 ng of Zika pRME alone or 500 ng of Zika pRME and 11 μg of opt-36γt in 30 μl of water delivered intramuscularly as described above.

Animal Challenge Studies

For the ZIKA challenge studies, IFNAR^{-/-} mice (n=12-14/group) were immunized once with 500 ng of ZIKA prME vaccine or 500 ng of pRME and 11 μg of opt-36γt. The mice were challenged with 1×105 PFU ZIKV-PR209 virus on day 15. Post challenge, the animals were weighed daily. In addition, they were observed for clinical signs of disease twice daily (decreased mobility; hunched posture; hind-limb knuckle walking (partial paralysis), paralysis of one hind limb or both hind limbs). The criteria for killing on welfare grounds consisted of 20% weight loss or paralysis in one or both hind limbs.

ELISpot Assay

Precoated anti-IFN-γ ninety-six well plates (MabTech, Cincinnati, Ohio) were used to quantify IFN-γ responses to vaccine. Spleens were isolated from mice either two weeks post vaccination. Single-cell suspensions of splenocytes were made by homogenizing and processing the spleens through a 40 μm cell strainer. Cells were then re-suspended in ACK Lysing buffer (Gibco™) for 5 min to lyse red blood cells before two washes with PBS and final re-suspension in RPMI complete media (RPMI 1640+10% FBS+1% penicillin-streptomycin). Two hundred thousand splenocytes were added to each well and stimulated overnight at 37° C. in 5% CO2 with R10 (negative control), concanavalin A (3 μg/ml; positive control), or 15-mer Zika pRME peptides overlapping by 11 amino acids (5 μg/ml; GenScript). After 18 hours of stimulation, the plates were washed and developed following manufacturer's protocol. The plates were then rinsed with distilled water and dried at room temperature overnight. Spots were counted by an automated ELISpot reader (Cellular Technology Ltd.).

Flow Cytometry

For intracellular cytokine staining, two million cells were stimulated in 96-well plates with overlapping peptide pools of Zika prME protein, media alone (negative control) and phorbol 12-myristate 13-acetate (PMA) and ionomycin (BD Biosciences, San Jose, Calif.) (positive control) for 6 hours at 37° C.+5% CO2 in the presence of GolgiPlug and GolgiStop™ (BD Biosciences). After 6 hours, cells were collected and stained in FACS buffer with a panel of surface antibodies containing live dead eFluor V450, FITC anti-CD4, Alexa Fluor 700 anti-CD44, and APC-Cy7 anti-CD8 for 30 min at 4° C. Cells were washed and then fixed with Foxp3/Transcription Factor Fixation/Permeabilization (ThermoFischer Scientific) for 20 min at 4° C. Cells were washed with Perm/Wash buffer before intracellular staining with PE-Cy7 anti-IL-2, PerCP-Cy5.5 anti-CD3α, PE anti-TNFα, and APC anti-IFNγ for 1 hour at 4° C. Cells were then washed with Perm/Wash buffer before suspension in Perm/Wash buffer and acquisition on a BD LSRII. All results were analyzed using FlowJo™ v.10.0 (TreeStar).

Statistical Analysis

Statistical analysis was performed using a one-way modified ANOVA with a Turkey post-hoc test for immunogenicity studies and Mantel-Cox test for challenge studies. All analysis was performed using GraphPad Prism Software (La Jolla, Calif.). Horizontal bars represent mean with error bars expressing the standard error.

### The Results of the Experiments are Now Described

Opt-36βt Coformulation Leads to Enhanced Immune Responses Against HIV Env DNA Vaccine Compared to Opt-36β

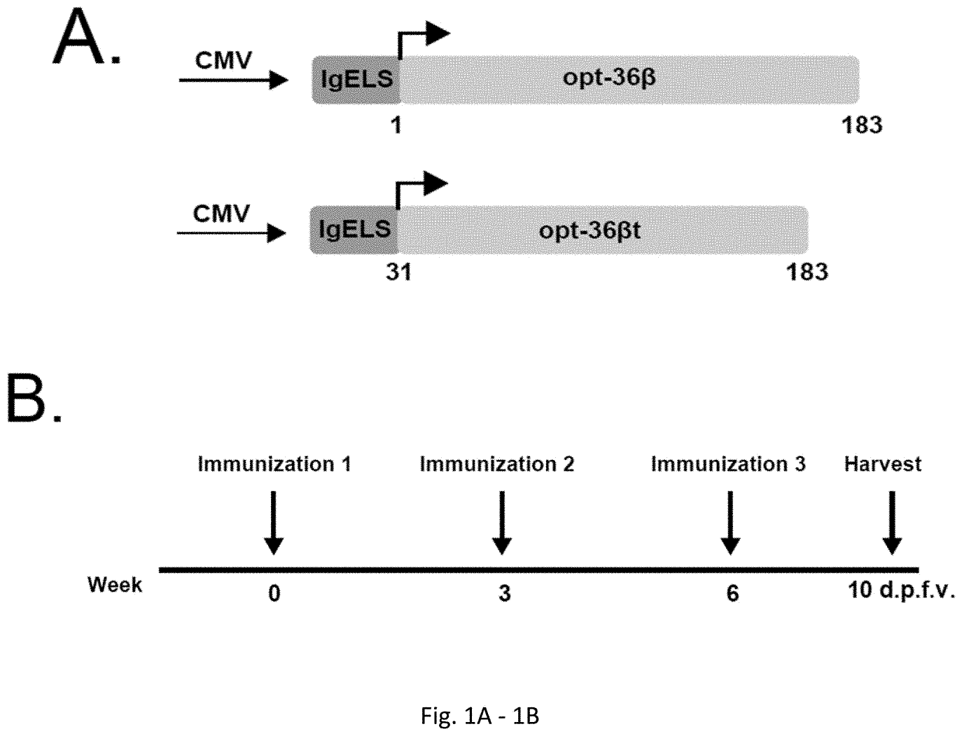

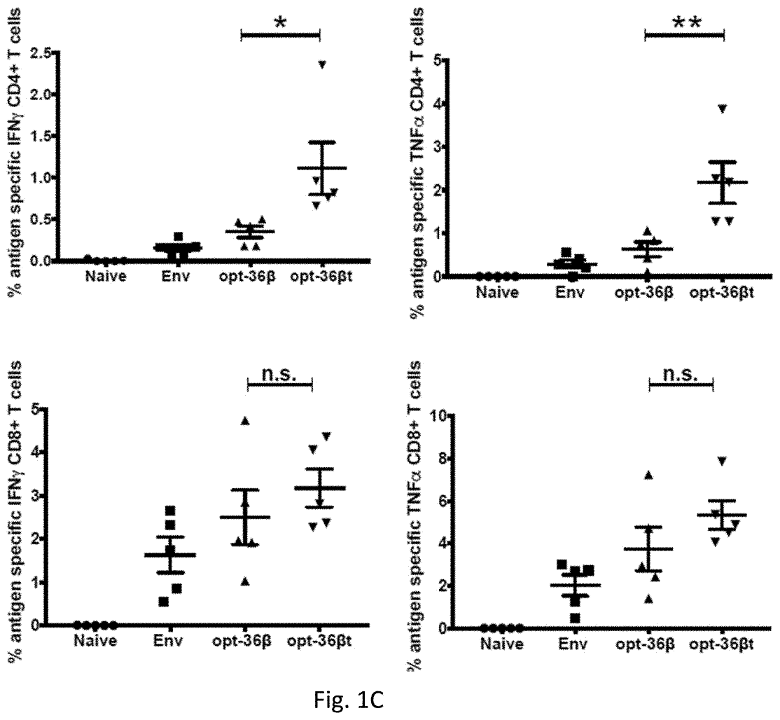

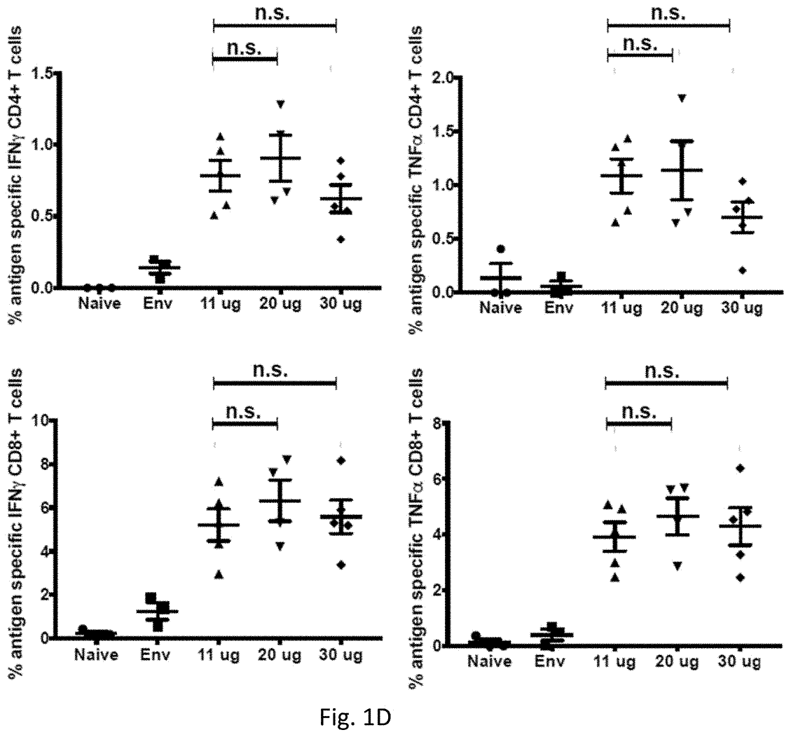

While the IL-36 family was discovered in 1999 (Gresnigt and van de Veerdonk, 2013, Semin Immunol, 25:458-65; Clavel et al., 2013, Joint Bone Spine, 80:449-53; Dinarello, 2013, Semin Immunol, 25:389-93; Catalan-Dibene et al., 2018, J Interferon Cytokine Res, 38:423-39), members of this family remain poorly understood and continue to be investigated. In the initial studies of their biology these cytokines either did not appear to have unique or robust activities and studies had to utilize very large quantities of recombinant protein to observe potential effects, thus limiting interest. With recent reports of IL-36 cytokines gaining activity only after N-terminal residue truncation (Towne et al., 2011, J Biol Chem, 286:42594-602; Henry et al., 2016, Cell Rep, 14:708-22; Clancy et al., 2016, FEBS Open Bio, 6:338-48), this study focused on whether truncation was important for an IL-36 in vivo produced gene adjuvant to impact immune profile of DNA vaccine antigens in a in vivo DNA vaccine model system. Initially experiments were performed with IL-36 beta, as IL-36 beta has been reported to amplify Th1 responses (Vigne et al., 2012, Blood, 120:3478-87), making it a potential cellular adjuvant candidate. Two DNA constructs were designed for these comparative studies, encoding either full length (opt-36β) or truncated (opt-36βt) IL-36 beta (FIG. 1A). A highly efficient IgE leader sequence was added to both of the sequences and they were RNA and codon optimized to enhance protein expression. C57BL/6 (B6) (n=5) mice were immunized with 2.5 μg of HIV Env DNA alone or with 11 μg opt-36β or opt-36βt, three times at three-week intervals using the 3P electrode driven by an adaptive electroporation CELLECTRA (EP) device (FIG. 1B). Spleens were harvested ten days post final vaccination for analysis antigen specific responses compared to control unadjuvanted HIV DNA vaccine constructs. Aa significant increase in the number of antigen specific CD4 T cells that secreted IFN-γ and TNF-α was observed in the group of animals whose vaccine had been adjuvanted by opt-36βt compared to opt-36β (FIG. 1C). There was a trend towards a similar pattern of enhancement for the antigen induced CD8 T cell responses, but in contrast to the CD4 T cell responses, this did not reach significance. A dosing study was next performed, focusing primarily on T cell induction to determine the optimal dose of opt-36βt. No significant difference was found in T cell response with the higher doses, and in fact there appeared to be a trend towards lowered immune response at the 30 μg dose (FIG. 1D). For the remainder of the studies going forward, the established 11 μg dose was maintained.

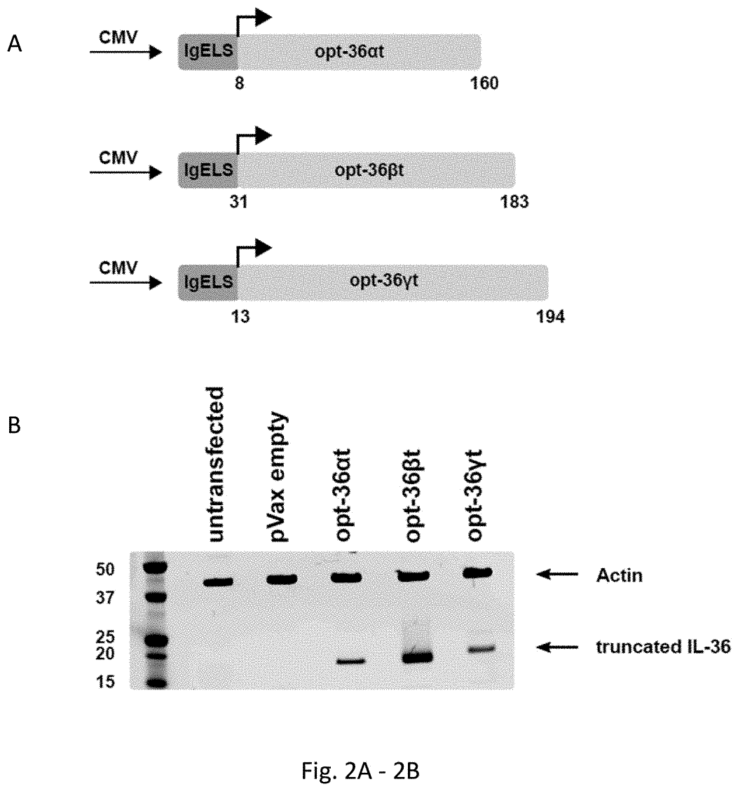

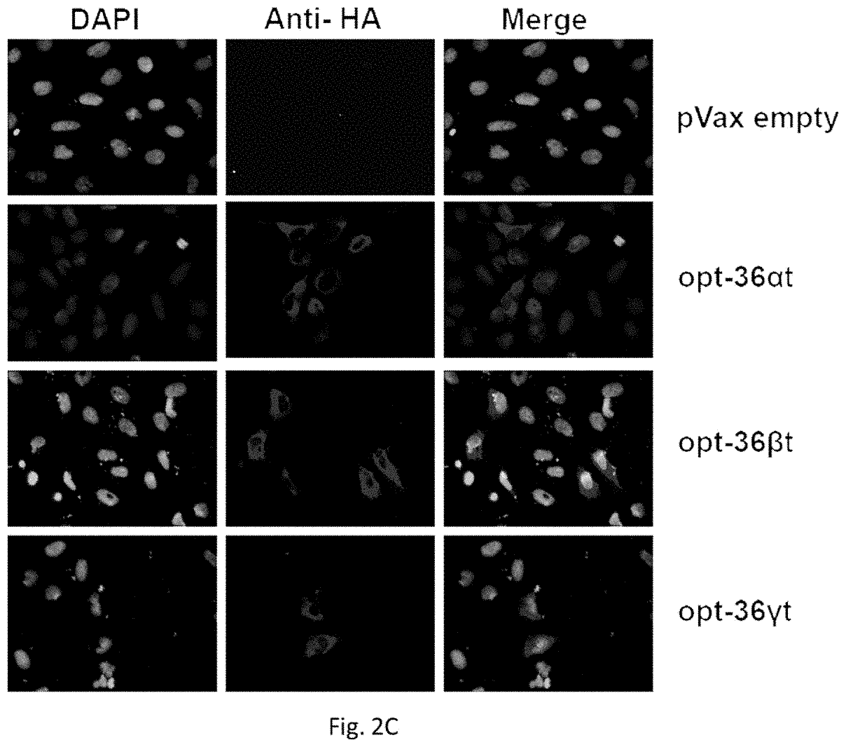

Given these results, the studies were expanded to examine the rest of the IL-36 family as truncated cytokines. In this regard, even less is known about IL-36 alpha or gamma than beta, so immune responses were evaluated in mice adjuvanted with each of the three cytokines in comparative studies. Truncated IL-36 alpha (opt-36αt) and IL-36 gamma (opt-36γt) were designed and modified as illustrated (FIG. 2A) (Andre et al., 1998, J Virol, 72:1497-503; Deml et al., 2001, J Virol, 75:10991-1001). Construct expression in vitro was confirmed using Western blot and immunofluorescence (FIG. 2B and FIG. 2C).

Opt-36βt and Opt-36γt Enhance Immune Responses Against HIV Env DNA Vaccine

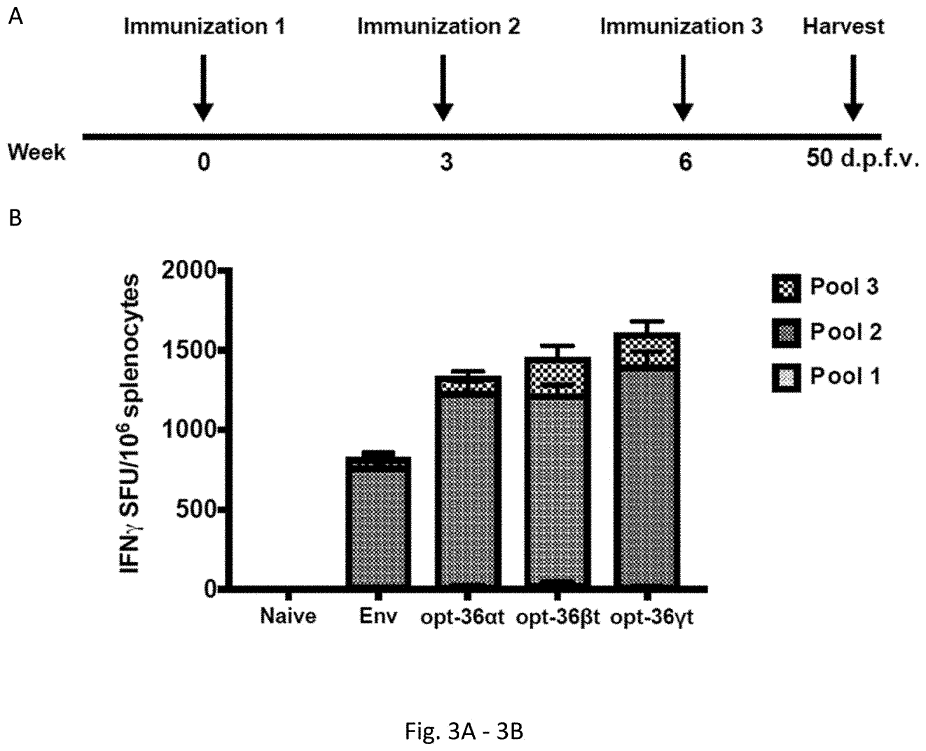

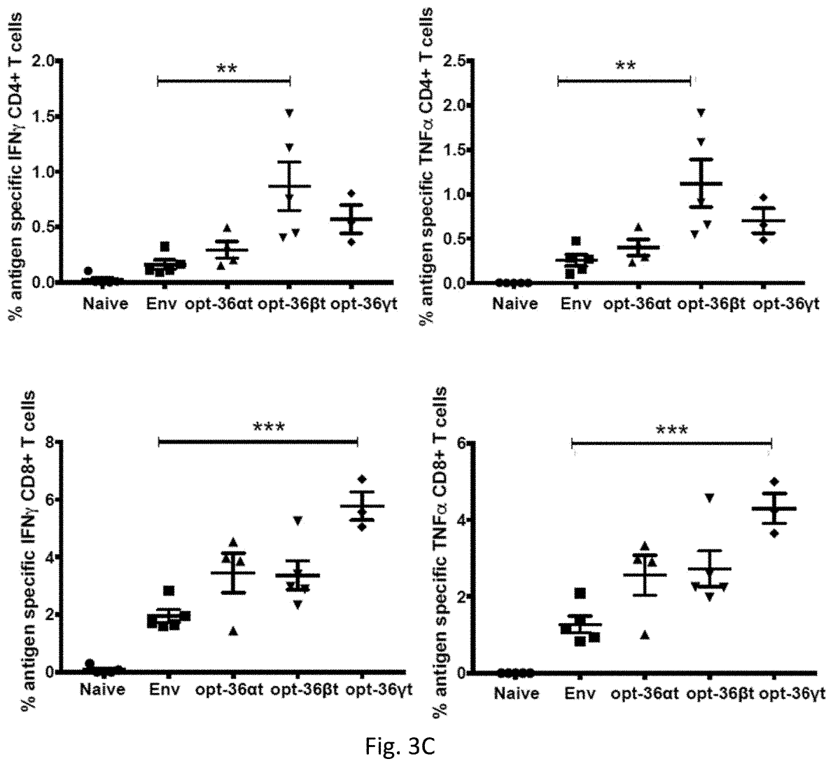

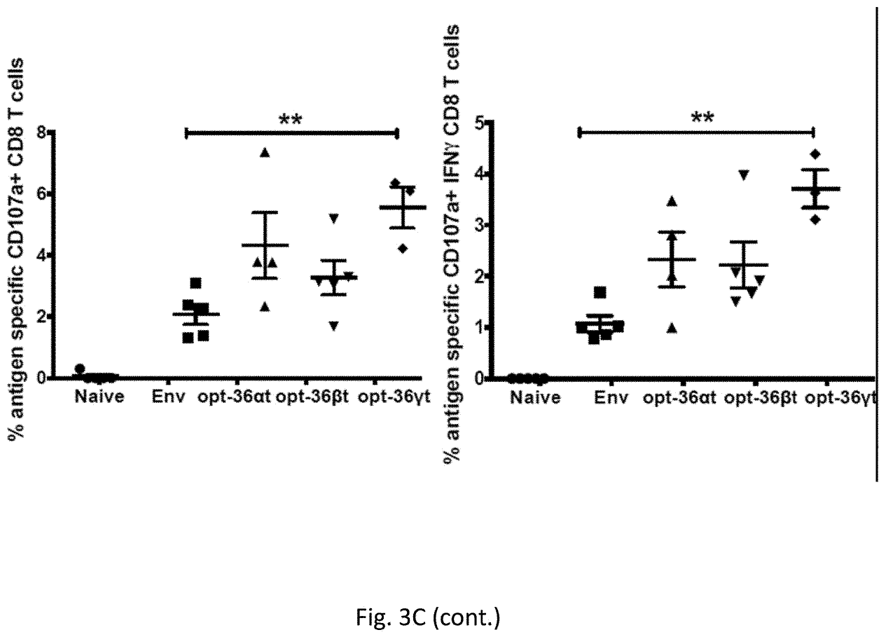

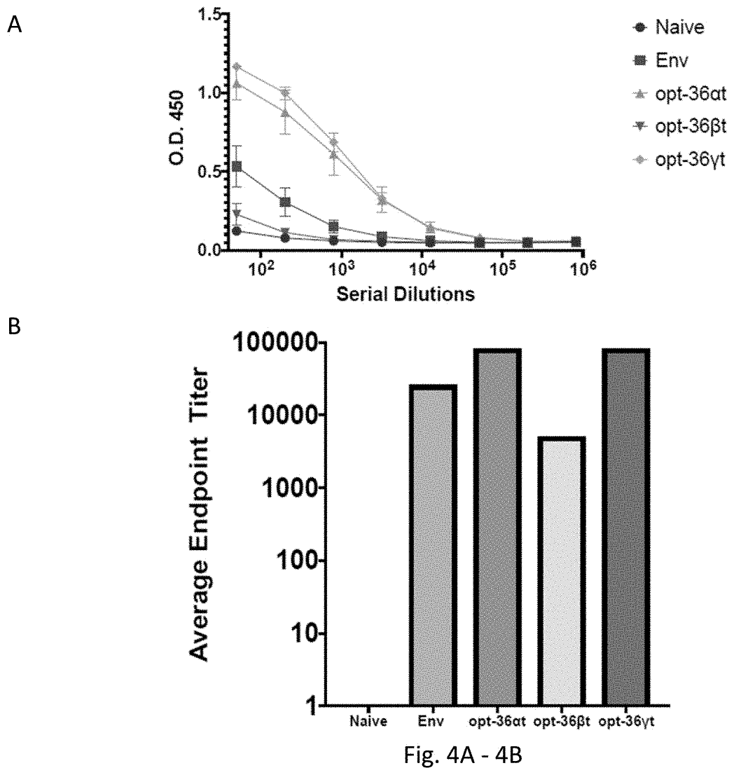

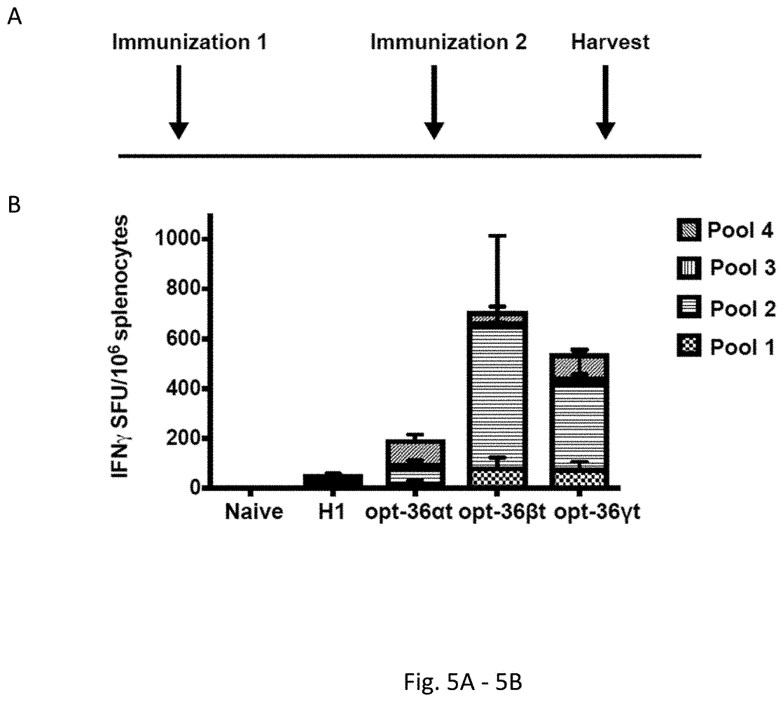

A major concern in the vaccine filed is the generation of vaccine candidates that can provide durable, long-term immune responses, and so it was examined whether immune responses following DNA vaccination would be maintained into memory. B6 mice (n=5/group) were immunized using 2.5 μg of HIV Env DNA alone or formulated with 11 μg of opt-36αt, opt-36βt, or opt-36γt three times at three-week intervals with CELLECTRA 3P electroporation (EP) (FIG. 3A). Spleens were harvested 50 days post final vaccination to analyze antigen specific responses. A quantitative ELISpot was performed to determine the number of Env specific IFN-γ secreting T cells that responded to vaccination (FIG. 3B). Mice immunized with the HIV vaccine alone produced an average of 775 spot forming units (SFU)/million splenocytes, while mice adjuvanted with opt-36αt, opt-36βt, and opt-36γt had on average 1242, 1460, and 1610 SFU/million splenocytes, supporting a potently enhanced response to the vaccine was driven by the adjuvants. Using intracellular cytokine staining it was observed again that mice adjuvanted with opt-36βt showed a significant number of CD4 T cells that expressed IFN-γ and TNF-α, compared to vaccine only. Similar to the results observed at an acute time point, mice adjuvanted with opt-36γt showed a 3-fold enhancement in the number of CD8 T cells that were expanded which expressed IFN-γ and TNF-α (FIG. 3C). Further, mice vaccinated with vaccine and opt-36γt had a significant number of CD107a+ IFN-γ CD8 T cells, suggesting the cytolytic potential of these CD8 cells in this model (FIG. 3C). The humoral response induced post vaccination was also examined, and it was observed that mice adjuvanted with opt-36αt and opt-36γt exhibited much higher antibody binding titers compared to mice immunized with Env alone (FIG. 4A and FIG. 4B). Of note, mice adjuvanted with opt-36βt exhibited suppressed antibody binding compared to vaccine alone.

Opt-36γt Enhances Humoral Immunity in Influenza DNA Vaccine Model

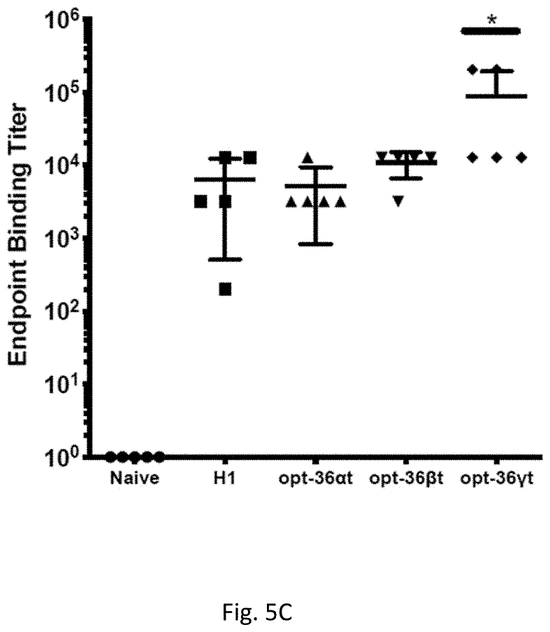

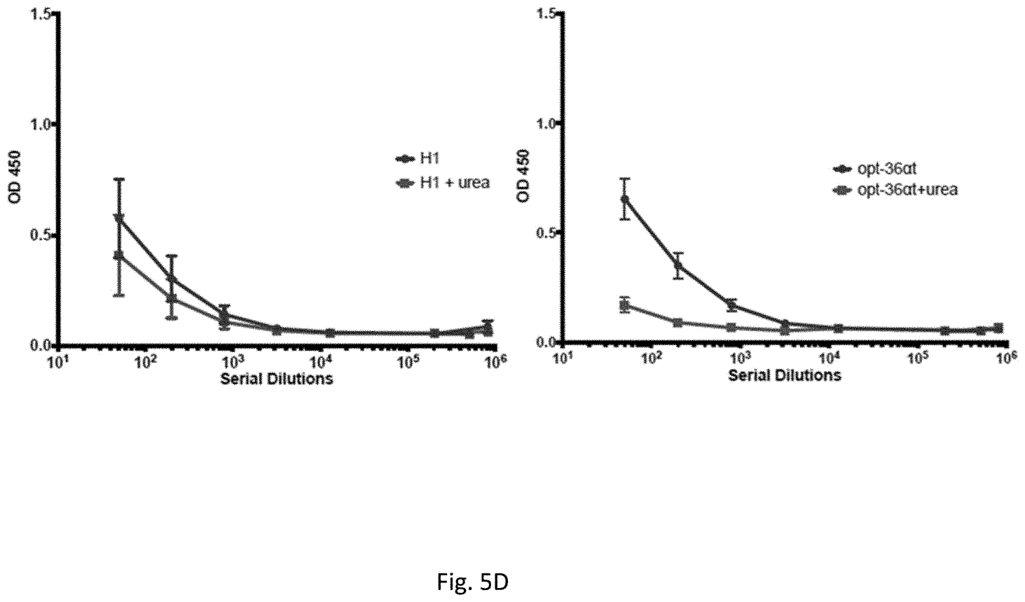

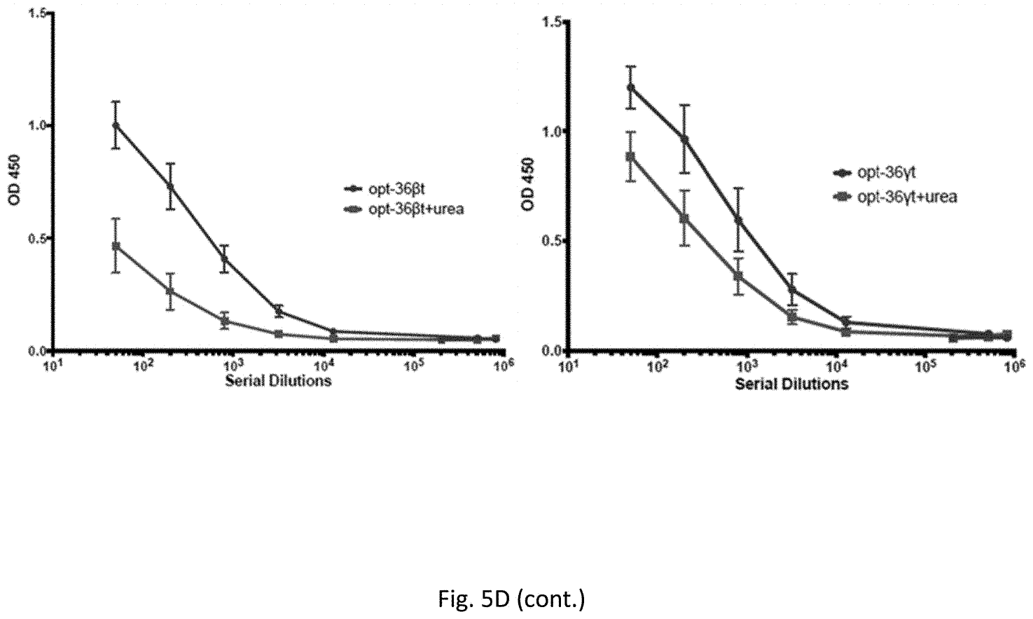

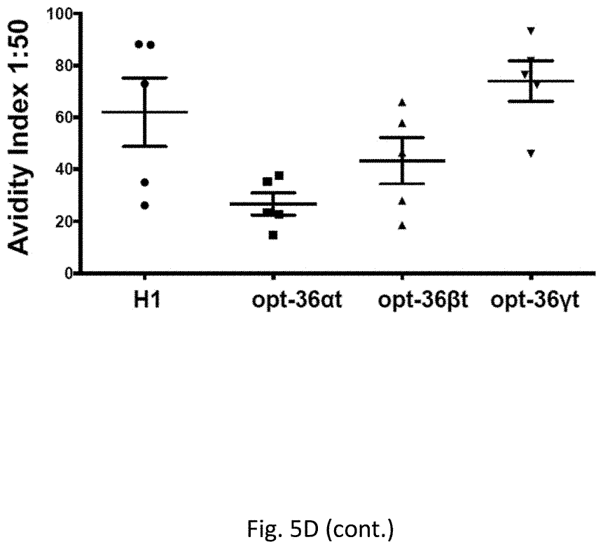

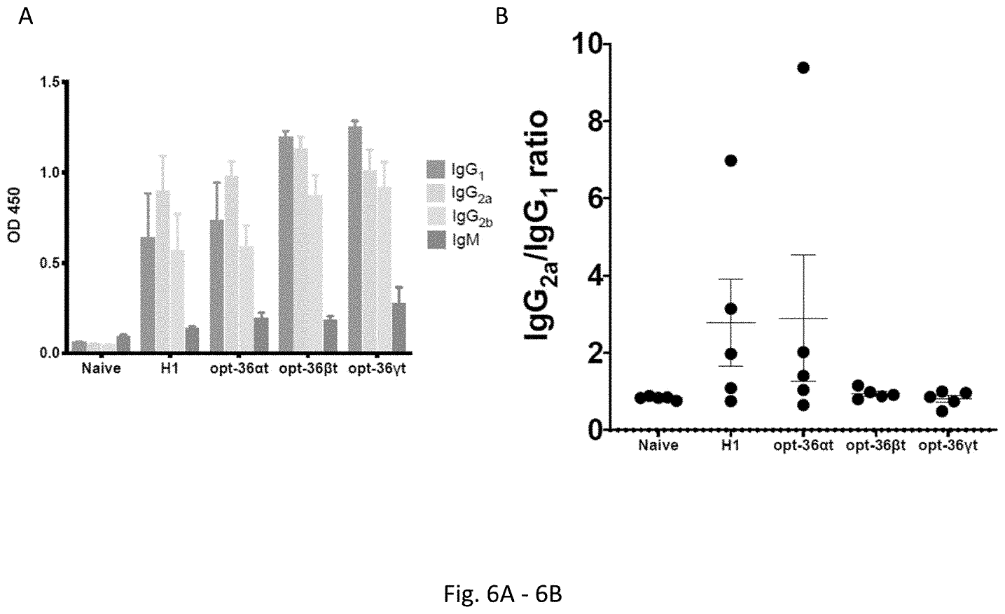

It was next sought to extend this finding to additional antigens with a different DNA vaccine antigen. Opt-36αt, opt-36βt, and opt-36γt's ability to impact immune responses was studied, driven by an H1 Syncon influenza DNA vaccine (Scott et al., 2015, Hum Vaccines Immunother, 11:1972-82). Given the potency of the adjuvant response in the previous studies, a two-dose regime was used to evaluate the vaccine-induced immune response in a dose sparing model. Balb/C mice (n=5/group) were immunized two times at two-week intervals with either 1 μg of H1 DNA alone or 1 μg of H1 and 11 μg of opt-36αt, opt-36βt, or opt-36γt by 3P electrode with EP (FIG. 5A). 10 days post final immunization, it was observed that by ELISpot, both opt-36βt and opt-36γt significantly enhanced cellular responses compared to the low dose vaccine alone (FIG. 5B), highlighting the potency of the adjuvants. Increased cellular responses were also observed in mice adjuvanted with opt-36αt. As antibodies are known to be critical for prevention from influenza infection, the binding antibody titers generated post vaccination were studied. Opt-36γt elicited significantly higher endpoint binding titers compared to naïve mice (FIG. 5C). The quality of the antibodies generated during vaccination was further examined by performing an ELISA based avidity test (Wise et al., 2015, J Virol, 89:9154) to examine strength of binding to a H1 influenza protein. Interestingly, mice adjuvanted with opt-36γt displayed both higher binding titers as well as exhibited a higher affinity binding antibody response for hemagglutinin protein, supporting induction of improved quality of antibodies by this novel adjuvant form (FIG. 5D). The isotypes of the antibodies generated post vaccination were examined, but no significant isotype switching was observed (FIG. 6).

Opt-36γt Enhances Cellular Immune Responses Induced by a Zika DNA Vaccine Resulting in Enhanced Protection Against Zika Challenge

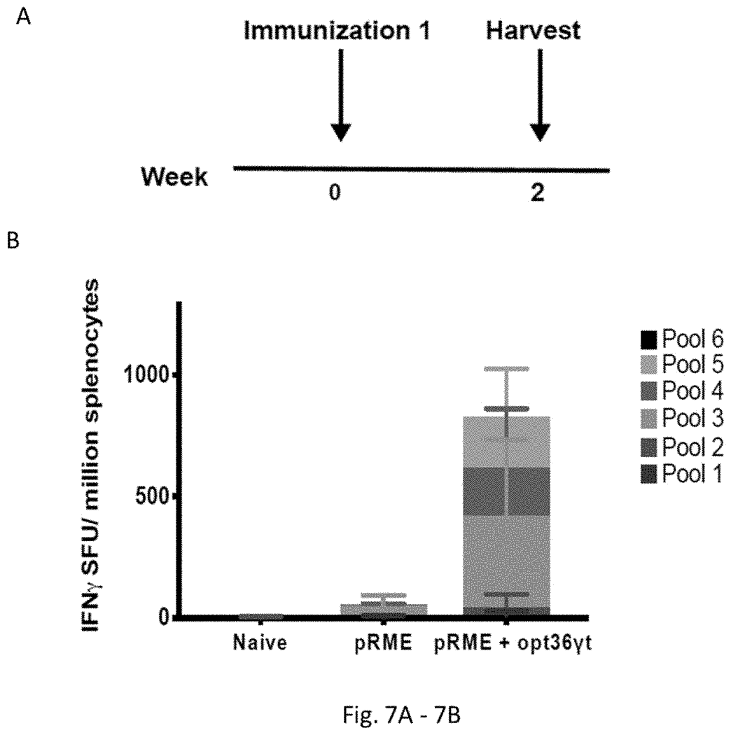

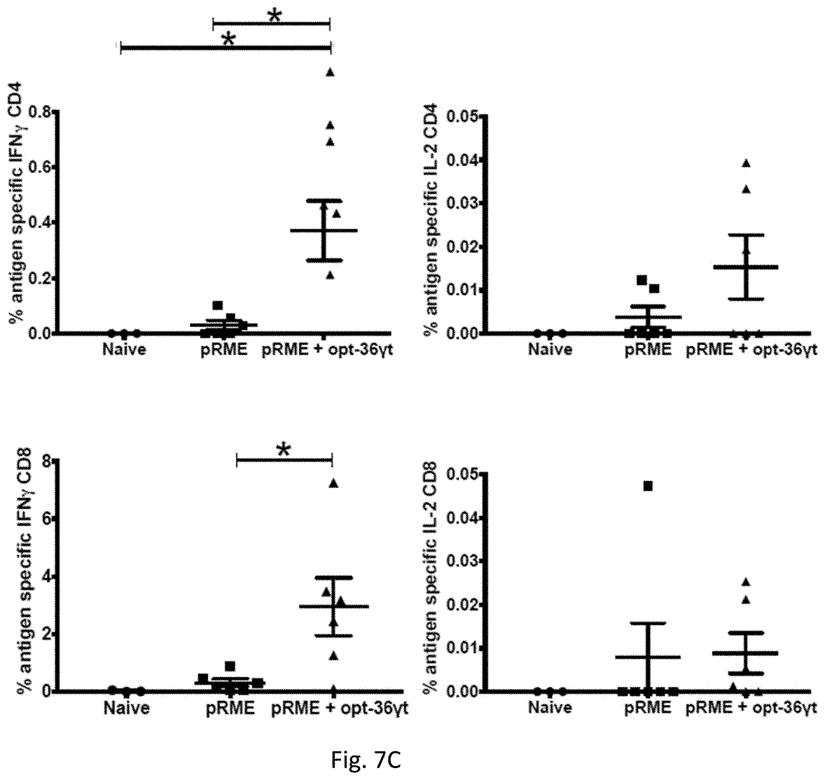

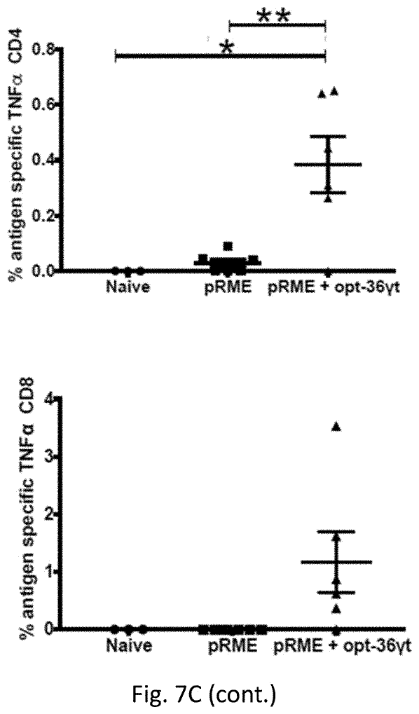

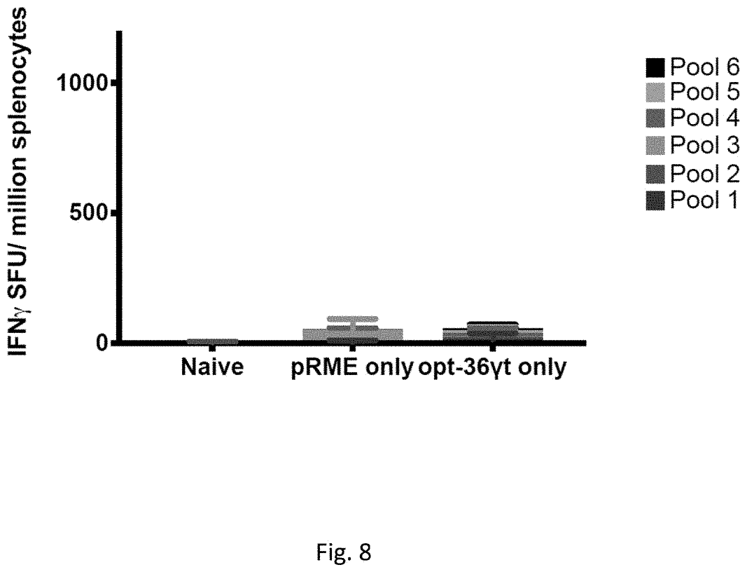

Based on the data generated in the two DNA vaccine models above, opt-36γt was studied in a well-characterized DNA viral challenge model. This allows for confirmation of the relevance of the improved immunity, and dose sparing potential driven by the opt-36γt adjuvant on protection of mice when using a suboptimal non-protective vaccine dose. IFNAR−/− mice (n=5-6 mice/group) were immunized just once with an exceptionally low 500 ng dose of Zika prME DNA vaccine alone, vs 11 μg of opt-36γt alone, or 500 ng of prME and 11 μg of opt-36γt. Two weeks following vaccination, spleens and blood were harvested (FIG. 7A). In ELISpot analysis, it was observed that mice immunized with vaccine only or with opt-36γt alone did not generate significant IFN-γ responses, but the combination of the two resulted in a synergy that generated potent IFN-γ responses resulting in 700 SFU/million splenocytes, which is in line with the response generated when mice are immunized with 25 μg of Zika pRME vaccine (Muthumani et al., 2016, Npj Vaccines, 1:16021) (FIG. 7B). This represents a 50× enhancement in cellular adjuvant activity. Immunization with opt-36γt alone did not generate significant cellular responses (FIG. 8). Using intracellular cytokine staining, it was observed that mice adjuvanted with opt-36γt exhibited increased IFN-γ and TNF-α expressing CD4 T cells as well as IFN-γ expressing CD8 T cells compared to the vaccine only treated mice (FIG. 7C). While there appeared to be a trend towards increased antibody titers in the mice adjuvanted with opt-36γt, overall antibody responses were very low in all groups, suggesting a need for an additional vaccine boost and studying higher vaccine doses to further detail the humoral immunity induced in this model.

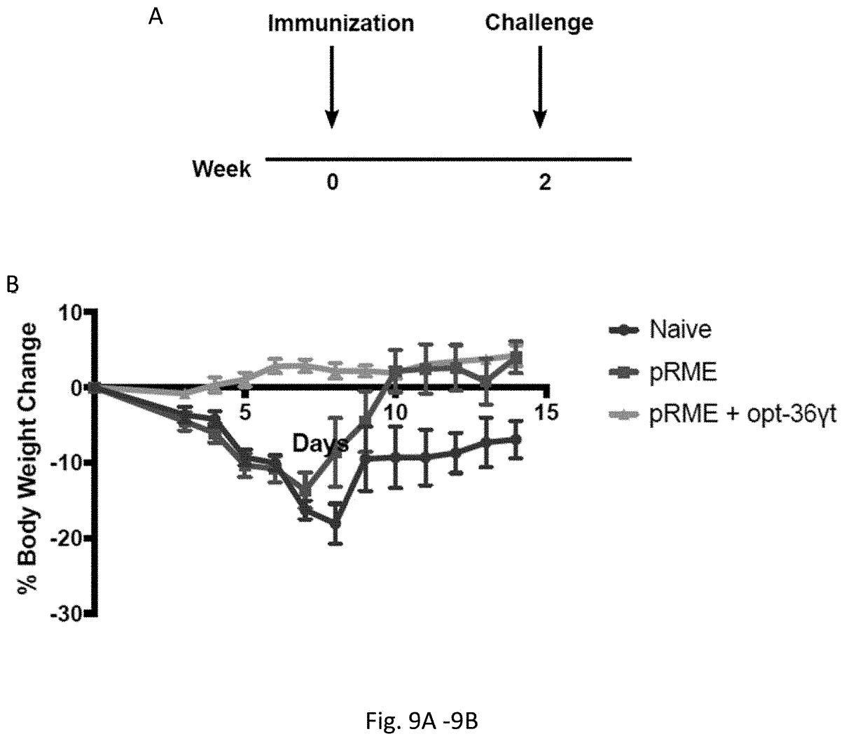

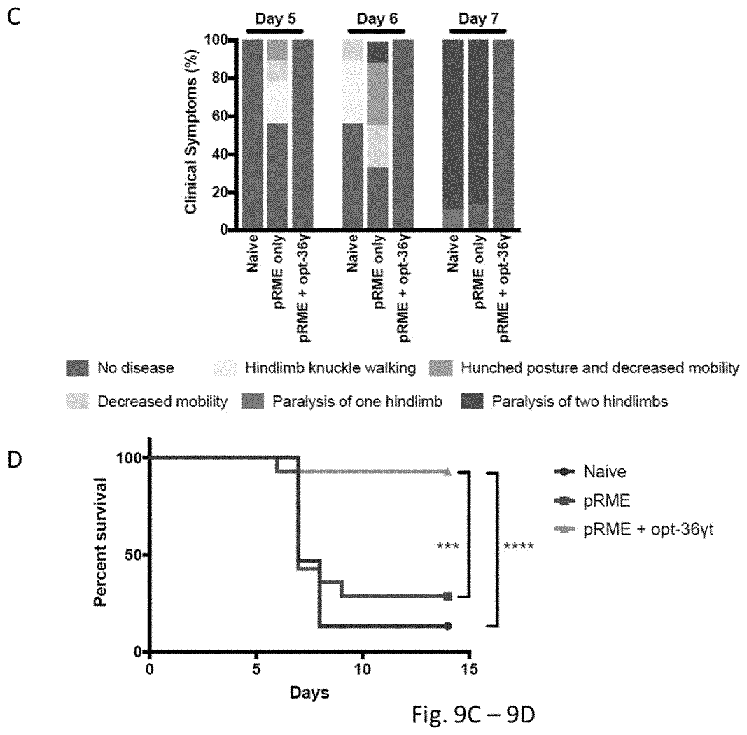

The study was repeated and this time a challenge was performed using IFNAR−/− mice (n=12-14/group) with a lethal dose of a validated Zika virus stock, strain PR 209. Challenge was performed two weeks after an immunization with either 500 ng of pRME alone or 500 ng of pRME and 11 μg of opt-36γt (FIG. 9A). The animals were followed for two weeks post challenge. One of the side effects typically observed that illustrate the morbidity effects of this Zika challenge in this mouse strain is weight loss (Muthumani et al., 2016, Npj Vaccines, 1:16021). Significant weight loss was observed in both the naïve and mice immunized with the suboptimal dose of the ZIKV-prME vaccine alone demonstrating substantial morbidity from the challenge (FIG. 9B). Naïve mice appeared to be the most impacted, with many mice losing up to 20% of their starting body weight, and with the low dose vaccine only group faring a bit better but still losing considerable weight. Strikingly, mice immunized with both prME along with opt-36γt were protected against weight loss, gaining weight during the course of the study. The mice were monitored for clinical symptoms during the challenge and observed that mice in both naïve and vaccine only group become progressively sicker (i.e. hunched posture and paralysis of hindlimbs) between days 5 and 7. However, the adjuvanted mice remain healthy and show no sign of disease following challenge (FIG. 9C). As animals succumb to disease they are sacrificed at predefined humane endpoints (Muthumani et al., 2016, Npj Vaccines, 1:16021). Dramatically, mice immunized with prME and opt-36γt exhibited a robust 92% survival rate, compared to a 28% survival rate for mice immunized with the prME and 13% survival rate for naïve mice (FIG. 9D). This data illustrates the significant benefit of the opt-36γt adjuvant in the context of this challenge model.

Sequences

SEQ ID NO:1 Human IL-36 alpha full length (nucleotide sequence, lacking IgE leader sequence and stop codons)

SEQ ID NO:2 Human IL-36 alpha full length (amino acid sequence, lacking IgE leader)

SEQ ID NO:3 Human IL-36 alpha truncated (nucleotide sequence, lacking IgE leader sequence and stop codons)

SEQ ID NO:4 Human IL-36 alpha truncated (amino acid sequence, lacking IgE leader)

SEQ ID NO:5 Human IL-36 beta full length (nucleotide sequence, lacking IgE leader sequence and stop codons)

SEQ ID NO:6 Human IL-36 beta full length (amino acid sequence, lacking IgE leader)

SEQ ID NO:7 Human IL-36 beta truncated (nucleotide sequence, lacking IgE leader sequence and stop codons)

SEQ ID NO:8 Human IL-36 beta truncated (amino acid sequence, lacking IgE leader)

SEQ ID NO:9 Human IL-36 gamma full length (nucleotide sequence, lacking IgE leader sequence and stop codons)

SEQ ID NO:10 Human IL-36 gamma full length (amino acid sequence, lacking IgE leader)

SEQ ID NO:11 Human IL-36 gamma truncated (nucleotide sequence, lacking IgE leader sequence and stop codons)

SEQ ID NO: 12 Human IL-36 gamma truncated (amino acid sequence, lacking IgE leader)

SEQ ID NO:13 Human IL-36 alpha full length (nucleotide sequence)

SEQ ID NO:14 Human IL-36 alpha full length (amino acid sequence)

SEQ ID NO:15 Human IL-36 alpha truncated (nucleotide sequence)

SEQ ID NO:16 Human IL-36 alpha truncated (amino acid sequence)

SEQ ID NO:17 Human IL-36 beta full length (nucleotide sequence)

SEQ ID NO:18 Human IL-36 beta full length (amino acid sequence)

SEQ ID NO:19 Human IL-36 beta truncated (nucleotide sequence)

SEQ ID NO:20 Human IL-36 beta truncated (amino acid sequence)

SEQ ID NO:21 Human IL-36 gamma full length (nucleotide sequence)

SEQ ID NO: 22 Human IL-36 gamma full length (amino acid sequence)

SEQ ID NO: 23 Human IL-36 gamma truncated (nucleotide sequence)

SEQ ID NO:24 Human IL-36 gamma truncated (amino acid sequence)

Murine IL-36 Alpha

SEQ ID NO: 25 Full length IL-36 alpha (nucleotide sequence) [opt-36α]

SEQ ID NO:26 Full length IL-36 alpha (amino acid sequence)

SEQ ID NO:27 Truncated IL-36 alpha (nucleotide sequence) [opt-36αt]

SEQ ID NO: 28 Truncated IL-36 alpha (amino acid sequence)

Murine IL-36 Beta

SEQ ID NO:29 Full length IL-36 beta (nucleotide sequence) [opt-36β]

SEQ ID NO:30 Full length IL-36 beta (amino acid sequence)

SEQ ID NO:31 Truncated IL-36 beta (nucleotide sequence) [opt-36βt]

SEQ ID NO:32 Truncated IL-36 beta (amino acid sequence)

Murine IL-36 Gamma

SEQ ID NO:33 Full length IL-36 gamma (nucleotide sequence) [opt-36γ]

SEQ ID NO: 34 Full length IL-36 gamma (amino acid sequence)

SEQ ID NO:35 Truncated IL-36 gamma (nucleotide sequence) [opt-36γt]

SEQ ID NO:36 Truncated IL-36 gamma (amino acid sequence)

SEQ ID NO:37 IgE leader sequence

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. An immunogenic composition comprising a nucleic acid molecule encoding an antigen and a nucleic acid molecule encoding an optimized IL-36 adjuvant.

2. The immunogenic composition of claim 1, wherein the IL-36 adjuvant is selected from the group consisting of optIL-36α, optIL-36β, optIL-36γ, a fragment of optIL-36α, a fragment of optIL-36β, a fragment of optIL-36γ, a variant of optIL-36α, a variant of optIL-36β, and a variant of optIL-36γ.

3. The immunogenic composition of claim 1, wherein the nucleic acid molecule encoding an optimized IL-36 adjuvant encodes an amino acid sequence selected from the group consisting of:
an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36;
an amino acid sequence having at least 95% identity to an amino acid sequence selected from the group consisting of SEQ ID NO:2, SEQ ID NO: 4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36;
a fragment of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36 comprising at least 60% of the full length sequence; and
a variant having at least 95% identity to a fragment of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36, wherein the fragment comprises at least 60% of the full length sequence.

4. The immunogenic composition of claim 1, wherein the nucleic acid molecule encoding an optimized IL-36 adjuvant comprises a nucleotide sequence selected from the group consisting of:
the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35;
a nucleotide having at least 95% identity to the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35;
a fragment of the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35 comprising at least 60% of the full length sequence; and
a variant having at least 95% identity to a fragment of the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35, wherein the fragment comprises at least 60% of the full length sequence.

5. The immunogenic composition of claim 1, wherein the antigen is selected from a group consisting of an HIV antigen, an influenza antigen, a ZIKA virus (ZIKV) antigen and a fragment thereof.

6. The immunogenic composition of claim 5, wherein the HIV antigen is selected from the group consisting of Env A, Env B, Env C, Env D, B Nef-Rev, Gag, and any combination thereof.

7. The immunogenic composition of claim 5, wherein the influenza antigen is selected from the group consisting of H1 HA, H2 HA, H3 HA, H5 HA, BHA antigen and any combination thereof.

8. The immunogenic composition of claim 5, wherein the ZIKV antigen includes a prME antigen.

9. The immunogenic composition of claim 1, further comprising a pharmaceutically acceptable excipient.

10. The immunogenic composition of claim 1, wherein the nucleic acid molecule encoding an antigen and the nucleic acid molecule encoding an optimized IL-36 adjuvant are expression vectors.

11. A method for increasing an immune response in a subject, the method comprising administering the immunogenic composition of claim 1 to the subject in need thereof.

12. The method of claim 11, wherein administering the immunogenic composition includes at least one of intramuscular administration and intradermal administration.

13. The method of claim 11, wherein administering the immunogenic composition includes electroporation.

14. The method of claim 11, wherein the increased immune response occurs in at least one of a skin tissue and a muscle tissue of the subject.

15. The method of claim 11, wherein the immune response in the subject is increased by about 75% to about 200%.

16. The method of claim 11, wherein the immune response in the subject is increased by at least about 1.5 fold.

17. A nucleic acid molecule comprising one or more nucleotide sequences encoding an optimized IL-36 adjuvant, wherein the IL-36 adjuvant is selected from the group consisting of optIL-36α, optIL-36β, optIL-36γ, a fragment of optIL-36α, a fragment of optIL-36β, a fragment of optIL-36γ, a variant of optIL-36α, a variant of optIL-36β, and a variant of optIL-36γ.

18. The nucleic acid molecule of claim 17, wherein the one or more nucleotide sequences encoding an optimized IL-36 adjuvant encodes an amino acid sequence selected from the group consisting of:
an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO: 20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36;
an amino acid sequence having at least 95% identity to an amino acid sequence selected from the group consisting of SEQ ID NO:2, SEQ ID NO: 4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36;
a fragment of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO: 18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36 comprising at least 60% of the full length sequence;
a variant having at least 95% identity to a fragment of an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, and SEQ ID NO:36, wherein the fragment comprises at least 60% of the full length sequence; and a combination thereof.

19. The nucleic acid molecule of claim 17 comprising one or more nucleotide sequences selected from the group consisting of:
the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35;
a nucleotide having at least 95% identity to the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO: 25, SEQ ID NO:27, SEQ ID NO:29, SEQ ID NO:31, SEQ ID NO:33, and SEQ ID NO:35;
a fragment of the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO: 17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35 comprising at least 60% of the full length sequence;
a variant having at least 95% identity to a fragment of the nucleotide sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, and SEQ ID NO:35, wherein the fragment comprises at least 60% of the full length sequence; and a combination thereof.

20. The nucleic acid molecule of claim 17, wherein the nucleic acid molecule is a plasmid.
